SEQUENCE LISTING (17 sequences)

SEQ ID NO: 1 - DNA, 1728 bp, mouse
atgagggcctggaccggctcctggaggtggatcatgctgatcctgttcgcctggggcaccctgctgttctacatcggcggccacctggtgagggacaacgaccaccccgaccactcctccagggagctgtccaagatcctggccaagctggagaggctgaagcagcagaacgaggacctgaggaggatggccgagtccctgaggatccccgagggccccatcgaccagggcaccgccaccggcagggtgagggtgctggaggagcagctggtgaaggccaaggagcagatcgagaactacaagaagcaggccaggaacggcctgggcaaggaccacgagatcctgaggaggaggatcgagaacggcgccaaggagctgtggttcttcctgcagtccgagctgaagaagctgaagcacctggagggcaacgagctgcagaggcacgccgacgagatcctgctggacctgggccaccacgagaggtccatcatgaccgacctgtactacctgtcccagaccgacggcgccggcgactggagggagaaggaggccaaggacctgaccgagctggtgcagaggaggatcacctacctgcagaaccccaaggactgctccaaggccaggaagctggtgtgcaacatcaacaagggctgcggctacggctgccagctgcaccacgtggtgtactgcttcatgatcgcctacggcacccagaggaccctgatcctggagtcccagaactggaggtacgccaccggcggctgggagaccgtgttcaggcccgtgtccgagacctgcaccgacaggtccggcctgtccaccggccactggtccggcgaggtgaacgacaagaacatccaggtggtggagctgcccatcgtggactccctgcaccccaggcccccctacctgcccctggccgtgcccgaggacctggccgacaggctgctgagggtgcacggcgaccccgccgtgtggtgggtgtcccagttcgtgaagtacctgatcaggccccagccctggctggagaaggagatcgaggaggccaccaagaagctgggcttcaagcaccccgtgatcggcgtgcacgtgaggaggaccgacaaggtgggcaccgaggccgccttccaccccatcgaggagtacatggtgcacgtggaggagcacttccagctgctggccaggaggatgcaggtggacaagaagagggtgtacctggccaccgacgaccccaccctgctgaaggaggccaagaccaagtactccaactacgagttcatctccgacaactccatctcctggtccgccggcctgcacaacaggtacaccgagaactccctgaggggcgtgatcctggacatccacttcctgtcccaggccgacttcctggtgtgcaccttctcctcccaggtgtgcagggtggcctacgagatcatgcagaccctgcaccccgacgcctccgccaacttccactccctggacgacatctactacttcggcggccagaacgcccacaaccagatcgccgtgtacccccacaagcccaggaccgaggaggagatccccatggagcccggcgacatcatcggcgtggccggcaaccactgggacggctactccaagggcatcaacaggaagctgggcaagaccggcctgtacccctcctacaaggtgagggagaagatcgagaccgtgaagtaccccacctaccccgaggccgagaagtga

SEQ ID NO: 2 - protein, 575 aa, human
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHEILRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLLRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPTLLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHKPRTEEEIPMEPGDIIGVAGNHWDGYSKGINRKLGKTGLYPSYKVREKIETVKYPTYPEAEK

SEQ ID NO: 3 - DNA, 1728 bp, Artificial Sequence
  Nucleic acid sequence of F83M modified enzyme, where positions 1093-1095, 1225-1227, and 1357-1359 in wild-type FUT8 gene have been changed
atgagggcctggaccggctcctggaggtggatcatgctgatcctgttcgcctggggcaccctgctgttctacatcggcggccacctggtgagggacaacgaccaccccgaccactcctccagggagctgtccaagatcctggccaagctggagaggctgaagcagcagaacgaggacctgaggaggatggccgagtccctgaggatccccgagggccccatcgaccagggcaccgccaccggcagggtgagggtgctggaggagcagctggtgaaggccaaggagcagatcgagaactacaagaagcaggccaggaacggcctgggcaaggaccacgagatcctgaggaggaggatcgagaacggcgccaaggagctgtggttcttcctgcagtccgagctgaagaagctgaagcacctggagggcaacgagctgcagaggcacgccgacgagatcctgctggacctgggccaccacgagaggtccatcatgaccgacctgtactacctgtcccagaccgacggcgccggcgactggagggagaaggaggccaaggacctgaccgagctggtgcagaggaggatcacctacctgcagaaccccaaggactgctccaaggccaggaagctggtgtgcaacatcaacaagggctgcggctacggctgccagctgcaccacgtggtgtactgcttcatgatcgcctacggcacccagaggaccctgatcctggagtcccagaactggaggtacgccaccggcggctgggagaccgtgttcaggcccgtgtccgagacctgcaccgacaggtccggcctgtccaccggccactggtccggcgaggtgaacgacaagaacatccaggtggtggagctgcccatcgtggactccctgcaccccaggcccccctacctgcccctggccgtgcccgaggacctggccgacaggctgctgagggtgcacggcgaccccgccgtgtggtgggtgtcccagttcgtgaagtacctgatcaggccccagccctggctggagaaggagatcgaggaggccaccaagaagctgggcttcaagcaccccgtgatcggcgtgcacgtggccaggaccgacaaggtgggcaccgaggccgccttccaccccatcgaggagtacatggtgcacgtggaggagcacttccagctgctggccaggaggatgcaggtggacaagaagagggtgtacctggccaccgccgaccccaccctgctgaaggaggccaagaccaagtactccaactacgagttcatctccgacaactccatctcctggtccgccggcctgcacaacaggtacaccgagaactccctgaggggcgtgatcctggccatccacttcctgtcccaggccgacttcctggtgtgcaccttctcctcccaggtgtgcagggtggcctacgagatcatgcagaccctgcaccccgacgcctccgccaacttccactccctggacgacatctactacttcggcggccagaacgcccacaaccagatcgccgtgtacccccacaagcccaggaccgaggaggagatccccatggagcccggcgacatcatcggcgtggccggcaaccactgggacggctactccaagggcatcaacaggaagctgggcaagaccggcctgtacccctcctacaaggtgagggagaagatcgagaccgtgaagtaccccacctaccccgaggccgagaagtga

SEQ ID NO: 4 - protein, 575 aa, Artificial Sequence
  Amino acid sequence of F83M modified enzyme, where the residues at positions 365, 409, and 453 in wild-type FUT8 protein have been chagned
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHEILRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLLRVHGDPAVWWVSQFVKYLIRPQPWLEKEIEEATKKLGFKHPVIGVHVARTDKVGTEAAFHPIEEYMVHVEEHFQLLARRMQVDKKRVYLATADPTLLKEAKTKYSNYEFISDNSISWSAGLHNRYTENSLRGVILAIHFLSQADFLVCTFSSQVCRVAYEIMQTLHPDASANFHSLDDIYYFGGQNAHNQIAVYPHKPRTEEEIPMEPGDIIGVAGNHWDGYSKGINRKLGKTGLYPSYKVREKIETVKYPTYPEAEK

SEQ ID NO: 5 - DNA, 1728 bp, Artificial Sequence
  Nucleic acids sequence of F8M1 modified enzyme, where positions 1105-1107 in wild-type FUT8 gene have been changed
atgagggcctggaccggctcctggaggtggatcatgctgatcctgttcgcctggggcaccctgctgttctacatcggcggccacctggtgagggacaacgaccaccccgaccactcctccagggagctgtccaagatcctggccaagctggagaggctgaagcagcagaacgaggacctgaggaggatggccgagtccctgaggatccccgagggccccatcgaccagggcaccgccaccggcagggtgagggtgctggaggagcagctggtgaaggccaaggagcagatcgagaactacaagaagcaggccaggaacggcctgggcaaggaccacgagatcctgaggaggaggatcgagaacggcgccaaggagctgtggttcttcctgcagtccgagctgaagaagctgaagcacctggagggcaacgagctgcagaggcacgccgacgagatcctgctggacctgggccaccacgagaggtccatcatgaccgacctgtactacctgtcccagaccgacggcgccggcgactggagggagaaggaggccaaggacctgaccgagctggtgcagaggaggatcacctacctgcagaaccccaaggactgctccaaggccaggaagctggtgtgcaacatcaacaagggctgcggctacggctgccagctgcaccacgtggtgtactgcttcatgatcgcctacggcacccagaggaccctgatcctggagtcccagaactggaggtacgccaccggcggctgggagaccgtgttcaggcccgtgtccgagacctgcaccgacaggtccggcctgtccaccggccactggtccggcgaggtgaacgacaagaacatccaggtggtggagctgcccatcgtggactccctgcaccccaggcccccctacctgcccctggccgtgcccgaggacctggccgacaggctgctgagggtgcacggcgaccccgccgtgtggtgggtgtcccagttcgtgaagtacctgatcaggccccagccctggctggagaaggagatcgaggaggccaccaagaagctgggcttcaagcaccccgtgatcggcgtgcacgtgaggaggaccgacgaggtgggcaccgaggccgccttccaccccatcgaggagtacatggtgcacgtggaggagcacttccagctgctggccaggaggatgcaggtggacaagaagagggtgtacctggccaccgacgaccccaccctgctgaaggaggccaagaccaagtactccaactacgagttcatctccgacaactccatctcctggtccgccggcctgcacaacaggtacaccgagaactccctgaggggcgtgatcctggacatccacttcctgtcccaggccgacttcctggtgtgcaccttctcctcccaggtgtgcagggtggcctacgagatcatgcagaccctgcaccccgacgcctccgccaacttccactccctggacgacatctactacttcggcggccagaacgcccacaaccagatcgccgtgtacccccacaagcccaggaccgaggaggagatccccatggagcccggcgacatcatcggcgtggccggcaaccactgggacggctactccaagggcatcaacaggaagctgggcaagaccggcctgtacccctcctacaaggtgagggagaagatcgagaccgtgaagtaccccacctaccccgaggccgagaagtga

SEQ ID NO: 6 - protein, 575 aa, Artificial Sequence
  Amino acid sequence of F8M1 modified, where the residue at position 369 in wild-type FUT8 protein has been changed
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHEILRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLLRVHGDPAVWWVSQFVKYLIRPQPWLEKEIEEATKKLGFKHPVIGVHVRRTDEVGTEAAFHPIEEYMVHVEEHFQLLARRMQVDKKRVYLATDDPTLLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHKPRTEEEIPMEPGDIIGVAGNHWDGYSKGINRKLGKTGLYPSYKVREKIETVKYPTYPEAEK

SEQ ID NO: 7 - DNA, 1728 bp, Artificial Sequence
  Nucleic acid sequence of F8M2 modified enzyme, where at positions 1225-1227 in wild-type FUT8 gene has been changed
atgagggcctggaccggctcctggaggtggatcatgctgatcctgttcgcctggggcaccctgctgttctacatcggcggccacctggtgagggacaacgaccaccccgaccactcctccagggagctgtccaagatcctggccaagctggagaggctgaagcagcagaacgaggacctgaggaggatggccgagtccctgaggatccccgagggccccatcgaccagggcaccgccaccggcagggtgagggtgctggaggagcagctggtgaaggccaaggagcagatcgagaactacaagaagcaggccaggaacggcctgggcaaggaccacgagatcctgaggaggaggatcgagaacggcgccaaggagctgtggttcttcctgcagtccgagctgaagaagctgaagcacctggagggcaacgagctgcagaggcacgccgacgagatcctgctggacctgggccaccacgagaggtccatcatgaccgacctgtactacctgtcccagaccgacggcgccggcgactggagggagaaggaggccaaggacctgaccgagctggtgcagaggaggatcacctacctgcagaaccccaaggactgctccaaggccaggaagctggtgtgcaacatcaacaagggctgcggctacggctgccagctgcaccacgtggtgtactgcttcatgatcgcctacggcacccagaggaccctgatcctggagtcccagaactggaggtacgccaccggcggctgggagaccgtgttcaggcccgtgtccgagacctgcaccgacaggtccggcctgtccaccggccactggtccggcgaggtgaacgacaagaacatccaggtggtggagctgcccatcgtggactccctgcaccccaggcccccctacctgcccctggccgtgcccgaggacctggccgacaggctgctgagggtgcacggcgaccccgccgtgtggtgggtgtcccagttcgtgaagtacctgatcaggccccagccctggctggagaaggagatcgaggaggccaccaagaagctgggcttcaagcaccccgtgatcggcgtgcacgtgaggaggaccgacaaggtgggcaccgaggccgccttccaccccatcgaggagtacatggtgcacgtggaggagcacttccagctgctggccaggaggatgcaggtggacaagaagagggtgtacctggccaccaaggaccccaccctgctgaaggaggccaagaccaagtactccaactacgagttcatctccgacaactccatctcctggtccgccggcctgcacaacaggtacaccgagaactccctgaggggcgtgatcctggacatccacttcctgtcccaggccgacttcctggtgtgcaccttctcctcccaggtgtgcagggtggcctacgagatcatgcagaccctgcaccccgacgcctccgccaacttccactccctggacgacatctactacttcggcggccagaacgcccacaaccagatcgccgtgtacccccacaagcccaggaccgaggaggagatccccatggagcccggcgacatcatcggcgtggccggcaaccactgggacggctactccaagggcatcaacaggaagctgggcaagaccggcctgtacccctcctacaaggtgagggagaagatcgagaccgtgaagtaccccacctaccccgaggccgagaagtga

SEQ ID NO: 8 - protein, 575 aa, Artificial Sequence
  Amino acid sequence of F8M2 modified enzyme, where the residue at position 409 in wild-type FUT8 protein has been chagned
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHEILRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLLRVHGDPAVWWVSQFVKYLIRPQPWLEKEIEEATKKLGFKHPVIGVHVRRTDKVGTEAAFHPIEEYMVHVEEHFQLLARRMQVDKKRVYLATKDPTLLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHKPRTEEEIPMEPGDIIGVAGNHWDGYSKGINRKLGKTGLYPSYKVREKIETVKYPTYPEAEK

SEQ ID NO: 9 - DNA, 1728 bp, Artificial Sequence
  Nucleic acid sequence of F8M3 modified enzyme, where positions 1405-1407 in wild-type FUT8 gene have been changed
atgagggcctggaccggctcctggaggtggatcatgctgatcctgttcgcctggggcaccctgctgttctacatcggcggccacctggtgagggacaacgaccaccccgaccactcctccagggagctgtccaagatcctggccaagctggagaggctgaagcagcagaacgaggacctgaggaggatggccgagtccctgaggatccccgagggccccatcgaccagggcaccgccaccggcagggtgagggtgctggaggagcagctggtgaaggccaaggagcagatcgagaactacaagaagcaggccaggaacggcctgggcaaggaccacgagatcctgaggaggaggatcgagaacggcgccaaggagctgtggttcttcctgcagtccgagctgaagaagctgaagcacctggagggcaacgagctgcagaggcacgccgacgagatcctgctggacctgggccaccacgagaggtccatcatgaccgacctgtactacctgtcccagaccgacggcgccggcgactggagggagaaggaggccaaggacctgaccgagctggtgcagaggaggatcacctacctgcagaaccccaaggactgctccaaggccaggaagctggtgtgcaacatcaacaagggctgcggctacggctgccagctgcaccacgtggtgtactgcttcatgatcgcctacggcacccagaggaccctgatcctggagtcccagaactggaggtacgccaccggcggctgggagaccgtgttcaggcccgtgtccgagacctgcaccgacaggtccggcctgtccaccggccactggtccggcgaggtgaacgacaagaacatccaggtggtggagctgcccatcgtggactccctgcaccccaggcccccctacctgcccctggccgtgcccgaggacctggccgacaggctgctgagggtgcacggcgaccccgccgtgtggtgggtgtcccagttcgtgaagtacctgatcaggccccagccctggctggagaaggagatcgaggaggccaccaagaagctgggcttcaagcaccccgtgatcggcgtgcacgtgaggaggaccgacaaggtgggcaccgaggccgccttccaccccatcgaggagtacatggtgcacgtggaggagcacttccagctgctggccaggaggatgcaggtggacaagaagagggtgtacctggccaccgacgaccccaccctgctgaaggaggccaagaccaagtactccaactacgagttcatctccgacaactccatctcctggtccgccggcctgcacaacaggtacaccgagaactccctgaggggcgtgatcctggacatccacttcctgtcccaggccgacttcctggtgtgcaccttcgtgtcccaggtgtgcagggtggcctacgagatcatgcagaccctgcaccccgacgcctccgccaacttccactccctggacgacatctactacttcggcggccagaacgcccacaaccagatcgccgtgtacccccacaagcccaggaccgaggaggagatccccatggagcccggcgacatcatcggcgtggccggcaaccactgggacggctactccaagggcatcaacaggaagctgggcaagaccggcctgtacccctcctacaaggtgagggagaagatcgagaccgtgaagtaccccacctaccccgaggccgagaagtga

SEQ ID NO: 10 - protein, 575 aa, Artificial Sequence
  Amino acid sequence of F83M modified enzyme, where the residue at position 469 in wild-type FUT8 protein has been changed
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHEILRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLLRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPTLLKEAKTKYSNYEFISDNSISWSAGLHNRYTENSLRGVILDIHFLSQADFLVCTFSVQVCRVAYEIMQTLHPDASANFHSLDDIYYFGGQNAHNQIAVYPHKPRTEEEIPMEPGDIIGVAGNHWDGYSKGINRKLGKTGLYPSYKVREKIETVKYPTYPEAEK

SEQ ID NO: 11 - DNA, 1665 bp, Artificial Sequence
  Nucleic acid sequence of F8D1 modified enzyme, where positions 1087-1149 in wild-type FUT8 gene have been deleted
atgagggcctggaccggctcctggaggtggatcatgctgatcctgttcgcctggggcaccctgctgttctacatcggcggccacctggtgagggacaacgaccaccccgaccactcctccagggagctgtccaagatcctggccaagctggagaggctgaagcagcagaacgaggacctgaggaggatggccgagtccctgaggatccccgagggccccatcgaccagggcaccgccaccggcagggtgagggtgctggaggagcagctggtgaaggccaaggagcagatcgagaactacaagaagcaggccaggaacggcctgggcaaggaccacgagatcctgaggaggaggatcgagaacggcgccaaggagctgtggttcttcctgcagtccgagctgaagaagctgaagcacctggagggcaacgagctgcagaggcacgccgacgagatcctgctggacctgggccaccacgagaggtccatcatgaccgacctgtactacctgtcccagaccgacggcgccggcgactggagggagaaggaggccaaggacctgaccgagctggtgcagaggaggatcacctacctgcagaaccccaaggactgctccaaggccaggaagctggtgtgcaacatcaacaagggctgcggctacggctgccagctgcaccacgtggtgtactgcttcatgatcgcctacggcacccagaggaccctgatcctggagtcccagaactggaggtacgccaccggcggctgggagaccgtgttcaggcccgtgtccgagacctgcaccgacaggtccggcctgtccaccggccactggtccggcgaggtgaacgacaagaacatccaggtggtggagctgcccatcgtggactccctgcaccccaggcccccctacctgcccctggccgtgcccgaggacctggccgacaggctgctgagggtgcacggcgaccccgccgtgtggtgggtgtcccagttcgtgaagtacctgatcaggccccagccctggctggagaaggagatcgaggaggccaccaagaagctgggcttcaagcaccccgtgatcggcgtggtgcacgtggaggagcacttccagctgctggccaggaggatgcaggtggacaagaagagggtgtacctggccaccgacgaccccaccctgctgaaggaggccaagaccaagtactccaactacgagttcatctccgacaactccatctcctggtccgccggcctgcacaacaggtacaccgagaactccctgaggggcgtgatcctggacatccacttcctgtcccaggccgacttcctggtgtgcaccttctcctcccaggtgtgcagggtggcctacgagatcatgcagaccctgcaccccgacgcctccgccaacttccactccctggacgacatctactacttcggcggccagaacgcccacaaccagatcgccgtgtacccccacaagcccaggaccgaggaggagatccccatggagcccggcgacatcatcggcgtggccggcaaccactgggacggctactccaagggcatcaacaggaagctgggcaagaccggcctgtacccctcctacaaggtgagggagaagatcgagaccgtgaagtaccccacctaccccgaggccgagaagtga

SEQ ID NO: 12 - protein, 548 aa, Artificial Sequence
  Amino acid sequence of F8D1 modified enzyme, where the residues at positions 365-386 in wild-type FUT8 protein have been deleted
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHEILRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLLRVHGDPAVWWVSQFVKYLIRPQPWLEKEIEEATKKLGFKHPVIGHFQLLARRMQVDKKRVYLATDDPTLLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHKPRTEEEIPMEPGDIIGVAGNHWDGYSKGINRKLGKTGLYPSYKVREKIETVKYPTYPEAEK

SEQ ID NO: 13 - DNA, 1119 bp, Mouse
atggcccacgcccccgcctcctgcccctcctccaggaactccggcgacggcgacaagggcaagcccaggaaggtggccctgatcaccggcatcaccggccaggacggctcctacctggccgagttcctgctggagaagggctacgaggtgcacggcatcgtgaggaggtcctcctccttcaacaccggcaggatcgagcacctgtacaagaacccccaggcccacatcgagggcaacatgaagctgcactacggcgacctgaccgactccacctgcctggtgaagatcatcaacgaggtgaagcccaccgagatctacaacctgggcgcccagtcccacgtgaagatctccttcgacctggccgagtacaccgccgacgtggacggcgtgggcaccctgaggctgctggacgccatcaagacctgcggcctgatcaactccgtgaagttctaccaggcctccacctccgagctgtacggcaaggtgcaggagatcccccagaaggagaccacccccttctaccccaggtccccctacggcgccgccaagctgtacgcctactggatcgtggtgaacttcagggaggcctacaacctgttcgccgtgaacggcatcctgttcaaccacgagtcccccaggaggggcgccaacttcgtgaccaggaagatctccaggtccgtggccaagatctacctgggccagctggagtgcttctccctgggcaacctggacgccaagagggactggggccacgccaaggactacgtggaggccatgtggctgatgctgcagaacgacgagcccgaggacttcgtgatcgccaccggcgaggtgcactccgtgagggagttcgtggagaagtccttcatgcacatcggcaagaccatcgtgtgggagggcaagaacgagaacgaggtgggcaggtgcaaggagaccggcaagatccacgtgaccgtggacctgaagtactacaggcccaccgaggtggacttcctgcagggcgactgctccaaggcccagcagaagctgaactggaagcccagggtggccttcgacgagctggtgagggagatggtgcaggccgacgtggagctgatgaggaccaaccccaacgcctga

SEQ ID NO: 14 - protein, 372 aa, Mouse
MAHAPASCPSSRNSGDGDKGKPRKVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAIKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFMHIGKTIVWEGKNENEVGRCKETGKIHVTVDLKYYRPTEVDFLQGDCSKAQQKLNWKPRVAFDELVREMVQADVELMRTNPNA

SEQ ID NO: 15 - DNA, 1119 bp, Artificial Sequence
  Nucleic acid sequence of GMD4M modified enzyme, where positions 463-465, 469-471, 535-537, and 547-549 in wild-type GMD gene have been changed
atggcccacgcccccgcctcctgcccctcctccaggaactccggcgacggcgacaagggcaagcccaggaaggtggccctgatcaccggcatcaccggccaggacggctcctacctggccgagttcctgctggagaagggctacgaggtgcacggcatcgtgaggaggtcctcctccttcaacaccggcaggatcgagcacctgtacaagaacccccaggcccacatcgagggcaacatgaagctgcactacggcgacctgaccgactccacctgcctggtgaagatcatcaacgaggtgaagcccaccgagatctacaacctgggcgcccagtcccacgtgaagatctccttcgacctggccgagtacaccgccgacgtggacggcgtgggcaccctgaggctgctggacgccatcaagacctgcggcctgatcaactccgtgaagttctaccaggcctccgcctccgccctgtacggcaaggtgcaggagatcccccagaaggagaccacccccttctaccccaggtcccccgccggcgccgccgccctgtacgcctactggatcgtggtgaacttcagggaggcctacaacctgttcgccgtgaacggcatcctgttcaaccacgagtcccccaggaggggcgccaacttcgtgaccaggaagatctccaggtccgtggccaagatctacctgggccagctggagtgcttctccctgggcaacctggacgccaagagggactggggccacgccaaggactacgtggaggccatgtggctgatgctgcagaacgacgagcccgaggacttcgtgatcgccaccggcgaggtgcactccgtgagggagttcgtggagaagtccttcatgcacatcggcaagaccatcgtgtgggagggcaagaacgagaacgaggtgggcaggtgcaaggagaccggcaagatccacgtgaccgtggacctgaagtactacaggcccaccgaggtggacttcctgcagggcgactgctccaaggcccagcagaagctgaactggaagcccagggtggccttcgacgagctggtgagggagatggtgcaggccgacgtggagctgatgaggaccaaccccaacgcctga

SEQ ID NO: 16 - protein, 372 aa, Artificial Sequence
  Amino acid sequence of GMD4M modified enzyme, where at positions 155, 157, 179, and 183 in wild-type GMD protein have been changed
MAHAPASCPSSRNSGDGDKGKPRKVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAIKTCGLINSVKFYQASASALYGKVQEIPQKETTPFYPRSPAGAAALYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFMHIGKTIVWEGKNENEVGRCKETGKIHVTVDLKYYRPTEVDFLQGDCSKAQQKLNWKPRVAFDELVREMVQADVELMRTNPNA

SEQ ID NO: 17 - DNA, 63 bp, Artificial Sequence
  Nucleic acid sequence deleted in F8D1 mutant gene
cacgtgaggaggaccgacaaggtgggcaccgaggccgccttccaccccatcgaggagtacatg

(19) United States
(12) Patent Application Publication
(10) Pub. No.: US 2021/0317499 A1
(43) Pub. Date: Oct. 14, 2021

(54) AFUCOSYLATED ANTIBODIES AND MANUFACTURE THEREOF

(71) Applicant: United BioPharma Inc., Hukou Township, Hsinchu County (TW)

(72) Inventors: Wen-Jiun PENG, Hukou Township (TW); Hui-Jung CHEN, Hukou Township (TW)

(73) Assignee: United BioPharma Inc., Hukou Township, Hsinchu County (TW)

(21) Appl. No.: 17/270,987

(22) PCT Filed: Aug. 29, 2018
(86) PCT No.: PCT/CN2018/102995

§ 371 Date: Feb. 24, 2021

(51) Int. Cl. C12P 21/00; C12N 9/10; C07K 16/00

(52) U.S. Cl. CPC C12P 21/00; C12N 9/1051; C07K 16/00; C12Y 204/01068; C07K 2317/732; C07K 2317/734; C07K 2317/41

(57) ABSTRACT

Provided methods for producing an afucosylated antibody, the afucosylated antibodies and composition thereof, and cells for producing antibodies. The method comprises introducing a nucleic acid encoding at least one modified enzyme of the fucosylation pathway to a host cell to produce the afucosylated antibody in the host cell. The afucosylated antibodies produced by the disclosed methods have increased ADCC activity and would not suppress their CDC and safety.

### FIELD OF THE INVENTION

The present disclosure relates to afucosylated proteins, including an afucosylated immunologically functional molecule having improved activity and therapeutic properties, and methods for making afucosylated proteins.

### BACKGROUND OF THE INVENTION

Glycoproteins mediate many essential functions in human beings including catalysis, signaling, cell-cell communication, and molecular recognition and association. Many glycoproteins have been exploited for therapeutic purposes and, during the last two decades, recombinant versions of naturally-occurring, secreted glycoproteins have been a major product of the biotechnology industry. Examples include erythropoietin (EPO), therapeutic monoclonal antibodies (therapeutic mAbs), tissue plasminogen activator (tPA), interferon-β, (IFN-β), granulocyte-macrophage colony stimulating factor (GM-CSF), and human chorionic gonadotropin (hCG).

Five classes of antibodies are present in mammals, i.e., IgM, IgD, IgG, IgA and IgE. Antibodies of human IgG class are mainly used in the diagnosis, prevention and treatment of various human diseases because of their long half lite in blood and functional characteristics, such as various effector functions and the like. The human IgG class antibody is further classified into the following 4 subclasses: IgG1, IgG2, IgG3 and IgG4. A large number of studies have been carried out for the antibody-dependent cellular cytotoxicity (ADCC) activity and complement-dependent cytotoxicity activity (CDC) as effector functions of the IgG class antibody, and it has been reported that antibodies of the IgG1 subclass have the greatest ADCC activity and CDC activity among the human IgG class antibodies.

Expression of ADCC activity and CDC activity of human IgG 1 subclass antibodies requires binding of the Fc region of antibody to an antibody receptor present on the surface of an effector cell, such as a killer cell, a natural killer cell, an activated macrophage or the like (hereinafter referred to as “FcγR”) and various complement components. It has been suggested that several amino acid residues in the second domain of the antibody hinge region and C region (hereinafter referred to as “Cγ2 domain”) and a sugar chain linked to the Cγ2 domain are also important for this binding reaction.

Reducing or inhibiting N-glycan fucosylation of antibodies, or Fc-fusion proteins, can enhance the ADCC activity. ADCC typically involves the activation of natural killer (NK) cells and is dependent on the recognition of antibody-coated cells by Fc receptors on the surface of the NK cell. Binding of the Fc domain to Fc receptors on the NK cells is affected by the glycosylation state of the Fc domain. In addition, the type of the N-glycan at the Fc domain also affects ADCC activity. Therefore, for an antibody composition, or a Fc-fusion protein composition, an increase of the relative amount of afucosyl N-glycans can enhance the binding affinity for an FcγRIII, or ADCC activity of the composition.

Several factors that can influence glycosylation, including the species, tissue, and cell type have all been shown to be important in the way that glycosylation occurs. In addition, the extracellular environment, through altered culture conditions such as serum concentration, may have a direct effect on glycosylation. Various methods have been proposed to alter the glycosylation pattern achieved in a particular host organism including introducing or overexpressing certain enzymes involved in oligosaccharide production (U.S. Pat. Nos. 5,017,335; 5,510,261). These schemes are not limited to intracellular methods (U.S. Pat. No. 5,278,299).

WO98/58964 describes antibody compositions wherein substantially all of the N-linked oligosaccharide is a G2 oligosaccharide. G2 refers to a biantennary structure with two terminal Gals and no NeuAcs. WO99/22764 refers to antibody compositions which are substantially free of a glycoprotein having an N-linked G1, G0, or G-1 oligosaccharide in its CH2 domain. G1 refers to a biantennary structure having one Gal and no NeuAcs, G0 refers to a biantennary structure wherein no terminal NeuAcs or Gals are present and G-1 refers to the core unit minus one GlcNAc.

WO00/61739 reports that 47% of anti-hIL-5R antibodies expressed by YB2/0 (rat myeloma) cells have α1-6 fucose-linked sugar chains, compared to 73% of those antibodies expressed by NSO (mouse myeloma) cells. The fucose relative ratio of α-hIL-5R antibodies expressed by various host cells was YB2/0<CHO/d<NSO.

WO02/31140 and WO03/85118 show that modification of fucose binding to a sugar chain can be controlled by using an RNAi to suppress the function of α1,6-fucosyltransferase. A process for producing an antibody composition using a cell, which comprises using a cell resistant to a lectin which recognizes a sugar chain in which 1-position of fucose is bound to 6-position of N-acetylglucosamine in the reducing end through α-bond in a complex N-glycoside-linked sugar chain.

The structure of sugar chain plays an important role in the effector function of human IgG1 subclass antibodies, and that it may be possible to prepare an antibody having greater effector function by changing the sugar chain structure. However, the structures of sugar chains are various and complex, and solution of the physiological roles of sugar chains would be insufficient and expensive. Thus, a method for producing an afucosylated antibody is required.

### REFERENCES

1. PAULSON, James, et al., “Process for controlling intracellular glycosylation of proteins” U.S. Pat. No. 5,047,335 (1991)
2. GOOCHEE, Charles F., et al., “Method of controlling the degradation of glycoprotein oligosaccharides produced by cultured Chinese hamster ovary cells” U.S. Pat. No. 5,510,261 (1996)
3. WONG, Chi-Huey, et al., “Method and composition for synthesizing siatylated glycosyl compounds” U.S. Pat. No. 5,278,299 (1994)
4. RAJU, T., Shantha, “Methods and compositions for galactosylated glycoproteins” WO/1998/058964 (1998)
5. RAJU, T., Shantha, “Methods and compositions comprising galactosylated glycoproteins” WO/1999/022764 (1999)
6. HANAI, Nobuo, et al., “Method for controlling the activity of immunologically functional molecule” WO/2000/061739 (2000)
7. KANDA, Yutaka, et al., “cells producing antibody compositions” WO/2002/031140 (2002)
8. BLUMBERG, R. S., et al., “Central airway administration for systemic delivery of therapeutics” WO 03/077834 (2002)
9. BLUMBERG; R. S., et al., “Central airway administration for systemic delivery of therapeutics” US Patent Application Publication 2003-0235536 (2003)
10. MÖSSENER, E., et al., “Increasing the efficacy of CD20 antibody therapy through the engineering of a new type II anti-CD20 antibody with enhanced direct and immune effector cell-mediated B-cell cytotoxicity” Blood 115, 4393-4402 (2010)
11. FERRARA, C., et al., “Unique carbohydrate-carbohydrate interactions are required for high affinity binding between FcγRIII and antibodies lacking core fucose” Proc Natl Acad Sci U.S.A. 108, 12669-12674 (2011)

### SUMMARY OF THE INVENTION

The present disclosure is directed to novel methods for producing afucosylated proteins, including afucosylated antibodies, having improved activity. The disclosure is also directed to afucosylated proteins produced by the disclosed methods and cells for producing the afucosylated proteins. The disclosed afucosylated antibodies have increased antibody-dependent cellular cytotoxicity (ADCC) activity compared to naturally-occurring fucosylated antibodies.

One aspect of the present disclosure relates to a method for producing an afucosylated protein, including an afucosylated antibody, in a host cell. The method of the present disclosure generally comprises introducing a nucleic acid encoding a modified enzyme of the fucosylation pathway to a host cell to inhibit the fucosylation of an antibody in the host cell. The modified enzyme can be derived from an enzyme in the fucosylation pathway. In certain embodiments, the modified enzyme can be derived from GDP-mannose 4,6-dehydratase (GMD), GDP-4-keto-6-deoxy-D-mannose epinierase-reductase (FX), and/or any of the fucosyltransferases (FUT1 to FUT12, POFUT1, and POFUT2). In some embodiments, the modified enzyme can be derived from GMD or FUT. In specific embodiments, the modified enzyme can be derived from α-1,6-fucosyltransferase (FUT8). The modified enzyme can inhibit the function of the host cell's naturally-occurring enzyme in the fucosylation pathway, which, in turn, inhibits the fucosylation of an antibody in the host cell.

In some embodiments, the method for producing an afucosylated protein, including an afucosylated antibody, comprises (a) providing a host cell, (b) introducing a nucleic acid encoding a modified enzyme of the fucosylation pathway to the host cell, and (c) producing an afucosylated protein in the host cell.

Another aspect of the present disclosure relates to an afucosylated protein, including an afucosylated antibody, produced by the method of the present disclosure. The afucosylated antibody has increased and improved activities compared to naturally-occurring fucosylated antibodies. In some embodiment, the antibody has increased and improved ADCC.

The present disclosure also relates to a cell for producing the afucosylated protein, including an afucosylated antibody.

A detailed description of the present disclosure is given in the following embodiments with reference to the accompanying drawings.

### BRIEF DESCRIPTION OF THE DRAWINGS

FIG. 1 is a Western blot profile of FUT8 proteins produced in RC79 cells (a stable clone expressing RITUXAN®) and recombinant RC79 cells expressing F83M, F8M1, F8M2, F8M3, or F8D1 mutant protein. The expression of glyceraldehyde-3-phosphate dehydrogenase (GAPDH) is used as a protein loading control. The expression level of FUT8 protein in RC79 recombinant cells expressing a mutant IFUT8 enzyme is similar to, or the same as, the expression level of FUT8 protein in the parent RC79 cell.

FIG. 2 is a flow cytometric analysis of RC79 recombinant cells expressing F83M mutant protein and RC79 parent cells. The peak with the dashed line represents RC79 recombinant cells expressing F83M stained with Rhodamine-LCA. The filled, grey peak represents RC79 recombinant cells expressing F83M without Rhodamine-LCA stain (negative control). The peak with the dotted line represents RC79 cells (parent cells that do not express F83M) stained with Rhodamine-LCA (positive control).

FIGS. 3a and 3b are graphs showing the results of ADCC assay. FIGS. 3a-3b illustrate the ADCC activity of RITUXAN® and afucosylated anti-CD20 mAb by PBMC cell from donor 1 (FIG. 3a) and donor 2 (FIG. 3b), respectively. The ADCC activity of afucosylated anti-CD20 mAb (clone R1) is significantly higher than RITUXAN®.

FIGS. 4a-4c are graphs showing the SPR sensorgrams of FcγRIIIa affinity assay with an SPR biosensor (BIACORE™ X100). His-tagged FcγRIIIa (1 μg/mL) and 5-80 nM afucosylated anti-CD20 mAb (FIG. 4a), 20-320 nM RITUXAN® (FIG. 4b), or 5-80 nM GAZYVA® (FIG. 4c) flowed through the anti-His antibody-immobilized CM5 chip sequentially at the flow rate of 30 μL/min. The afucosylated anti-CD20 mAb (clone R1) had a stronger affinity to FcγRIIIa than RITUXAN® and GAZYVA®.

FIG. 5 is graph showing an CDC activity of RITUXAN® and afucosylated anti-CD20 mAb. The CDC activity of afucosylated anti-CD20 mAb (clone R1) was comparable with that of RITUXAN®.

FIG. 6 is a graph showing the tumor volume of mice treated with saline (vehicle), RITUXAN®, or afucosylated anti-CD20 mAb (clone R1). Data points indicate mean±SD of tumor volume (n=5 in each group). The anti-tumor efficacy of afucosylated anti-CD20 mAb (clone R1) is significantly higher than RITUXAN®.

FIG. 7 is a graph showing the weight of tumor collected from mice treated with saline (vehicle), RITUXAN®, or afucosylated anti-CD20 mAb (clone R1). The tumor weight of the mice treated with afucosylated anti-CD20 antibody (R1 clone) is significantly lighter than RITUXAN® and vehicle group.

FIG. 8 is a graph showing the body weight of mice treated with saline (vehicle), RITUXAN®, or afucosylated anti-CD20 mAb (clone R1). Data points indicate mean±SD of tumor volume (n=5 in each group).

### DETAILED DESCRIPTION OF THE INVENTION

The present disclosure is directed to novel methods for producing afucosylated antibodies with improved activity. The disclosure is also directed to afucosylated antibodies produced by the disclosed methods and cells for producing the afucosylated antibodies. The disclosed afucosylated antibodies have increased antibody-dependent cellular cytotoxicity (ADCC) activity compared to naturally-occurring fucosylated antibodies.

The following is a detailed description provided to aid those skilled in the art in practicing the present invention. Those of ordinary skill in the art would understand that modifications or variations of the embodiments expressly described herein, which do not depart from the spirit or scope of the information contained herein, are encompassed by the present disclosure. The terminology used in the description is for describing particular embodiments only and is not intended to be limiting of the invention. The section headings used below are for organizational purposes only and are not to be construed as limiting the subject matter described.

All publications, patent applications, patents, figures and other references, including portions thereof, mentioned herein are incorporated by reference in their entireties as if disclosed and recited completely in the specification.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all amino acid sizes, and all molecular weight or molecular mass values, given for polypeptides are approximate, and are provided for description. Although methods and materials similar or equivalent to those described herein can be used in the practice or testing of the disclosed method, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### 1. Inhibiting Fucosylation in a Cell

One aspect of the present disclosure relates to a method for inhibiting or reducing fucosylation in a cell.

a. Host Cells

Any appropriate host cell can be used to produce afucosylated antibodies, including a host cell derived from yeast, insect, amphibian, fish, reptile, bird, mammal, or human, or a hybridoma cell. The host cell can be an unmodified cell or cell line, or a cell line that has been genetically modified (e.g., to facilitate production of a biological product). In some embodiments, the host cell is a cell line that has been modified to allow for growth under desired conditions, such as in serum-free media, in cell suspension culture, or in adherent cell culture.

A mammalian host cell can be advantageous to use for antibodies intended for administration to humans. In some embodiments, the host cell is a Chinese hamster ovary (CHO) cell, which is a cell line used for the expression of many recombinant proteins. Additional mammalian cell lines commonly used for the expression of recombinant proteins include 293HEK cells, HeLa cells, COS cells, NIH/3T3 cells, Jurkat cells, NSO cells, and HUVEC cells. In other embodiments, the host cell is a recombinant cell which expresses an antibody.

Examples of human cell lines useful in methods provided herein include the cell lines 293T (embryonic kidney), 786-0 (renal), A498 (renal), A549 (alveolar basal epithelial), ACHN (renal), BT-549 (breast), BxPC-3 (pancreatic), CAKI-1 (renal), Capan-i (pancreatic), CCRF-CEM (leukemia), COLO 205 (colon), DLD-1 (colon), DMS 114 (small cell lung), DU145 (prostate), EKVX (non-small cell lung), HCC-2998 (colon), HCT-15 (colon), HCT-1 16 (colon), HT29 (colon), S IT-1080 (fibrosarcoma), HEK 293 (embryonic kidney), HeLa (cervical carcinoma), HepG2 (hepatocellular carcinoma), HL-60(TB) (leukemia), HOP-62 (non-small cell lung), HOP-92 (non-small cell lung), HS 578T (breast), HT-29 (colon adenocarcinoma), IG-OV1 (ovarian), IMR32 (neuroblastoma), Jurkat (T lymphocyte), K-562 (leukemia), KM 12 (colon), KM20L2 (colon), LANS (neuroblastoma), LNCap.FGC (Caucasian prostate adenocarcinoma), LOX IMV1 (melanoma), LXFL 529 (non-small cell lung), M 14 (melanoma), M19-MEL (melanoma), MALME-3M (melanoma), MCFIOA (mammary epithelial), MCI '7 (mammary), MDA-MB-453 (mammary epithelial), MDA-MB-468 (breast), MDA-MB-231 (breast), MDA-N (breast), MOLT-4 (leukemia), NC1/ADR-RES (ovarian), NCI-1122.0 (non-small cell lung), NCI-H23 (non-small cell lung), NCI-H322M (non-small cell lung), NCI-H460 (non-small cell lung), NCI-H522 (non-small cell lung), OVCAR-3 (ovarian), QVCAR-4 (ovarian), OVCAR-5 (ovarian), OVCAR-8 (ovarian), P388 (leukemia), P388/ADR (leukemia), PC-3 (prostate), PERC6® (E1-transformed embryonal retina), RPMI-7951 (melanoma), RPMI-8226 (leukemia), RXF 393 (renal), RXF-631 (renal), Saos-2 (bone), SF-268 (CNS), SF-295 (CNS), SF-539 (CNS), SHP-77 (small cell lung), SH-SY5Y (neuroblastoma), SK-BR3 (breast), SK-MEL-2 (melanoma), SK-MEL-5 (melanoma), SK-MEL-28 (melanoma), SK-OV-3 (ovarian), SN12K1 (renal), SN12C (renal), SNB-19 (CNS), SNB-75 (CNS) SNB-78 (CNS), SR (leukemia), SW-620 (colon), T-47D (breast), THP-1 (monocyte-derived macrophages), TK-10 (renal), U87 (glioblastoma), U293 (kidney), U251 (CNS), UACC-257 (melanoma), UACC-62 (melanoma), UO-31 (renal), W138 (lung), and XF 498 (CNS).

Examples of non-human primate cell lines useful in methods provided herein include the cell lines monkey kidney (CVI-76), African green monkey kidney (VERO-76), green monkey fibroblast (COS-1), and monkey kidney (CVI) cells transformed by SV40 (COS-7). Additional mammalian cell lines are known to those of ordinary skill in the art and are catalogued at the American Type Culture Collection (ATCC) catalog (Manassas, Va.).

b. Modifying an Enzyme in the Fucosylation Pathway

Afucosylated antibodies of the present disclosure can be produced in a host cell in which the fucosylation pathway has been altered in a way that reduces or inhibits fucosylation of proteins.

i. Modified Enzymes

The phrase “modified enzyme” as used herein, refers to a protein derived from a naturally-occurring, or wild-type, enzyme in the fucosylation pathway that has been altered in a way that changes or destroys the natural enzymatic activity of the protein after modification. A modified enzyme is capable of inhibiting or interfering with its wild-type counterpart to change, inhibit, or reduce the activity of the wild-type enzyme in a host cell.

A modified enzyme can be produced by altering the naturally-occurring enzyme, for example, by changing the overall protein charge, covalently attaching a chemical or protein moiety, introducing amino acid substitutions, insertions, and/or deletions, and/or any combination thereof. In some embodiments, the modified enzyme has amino acid substitutions, additions, and/or deletions compared to its naturally-occurring enzyme counterpart. In some embodiments, the modified enzyme has between one to about twenty amino acid substitutions, additions, and/or deletions compared to its naturally-occurring counterpart. The amino acid substitution, addition, and insertion can be accomplished with natural or non-natural amino acids. Non-naturally occurring amino acids include, but are not limited to, ε-N Lysine, β-alanine, ornithine, norleucine, norvaline, hydroxyproline, thyroxine, γ-amino butyric acid, homoserine, citrulline, aminobenzoic acid, 6-Aminocaproic acid (Aca; 6-Aminohexanoic acid), hydroxyproline, mercaptopropionic acid (MPA), 3-nitro-tyrosine, pyroglutamic acid, and the like. Naturally-occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

The modified enzyme can be derived from any naturally-occurring enzyme in the fucosylation pathway. For example, the modified enzyme can be derived from GDP-mannose 4,6-dehydratase (GMD), GDP-4-keto-6-deoxy-D-mannose epinierase-reductase (FX), and/or any of the fucosyltransferases, including: galactoside 2-alpha-L-fucosyltransferase 1 (FUT1), galactoside 2-alpha-L-fucosyltransferase 2 (FUT2), galactoside 3(4)-L-fucosyltransferase (FUT3), alpha (1,3) fucosyltransferase, myeloid-specific (FUT4), alpha-(1,3)-fucosyltransferase (FUT5), alpha-(1,3)-fucosyltransferase (FUT6), alpha-(1,3)-fucosyltransferase (FUT7), alpha-(1,6)-fucosyltransferase (FUT8), alpha-(1,3)-fucosyltransferase (FUT9), protein O-fucosyltransferase 1 (POFUT1), protein O-fucosyltransferase 2 (POFUT2).

In some embodiments, more than one enzyme in the fucosylation pathway is modified. In certain embodiments, the modified enzyme is derived from GMD, FX, and/or FUT8.

ii. Nucleic Acids Encoding a Modified Enzyme

Afucosylated antibodies of the present disclosure can be produced in a host cell in which the fucosylation pathway has been altered in a way that reduces or inhibits fucosylation of proteins.

In some embodiments, the fucosylation pathway of a host cell is altered by introducing to the cell a nucleic acid that encodes a modified enzyme in the fucosylation pathway. For example, a nucleic acid encoding the modified enzyme can be inserted into an expression vector and transfected into a host cell. The nucleic acid molecule encoding the modified enzyme can be transiently introduced into the host cell, or stably integrated into the genome of the host cell. Standard recombinant DNA methodologies may be used to produce a nucleic acid that encodes the modified enzyme, incorporate the nucleic acid into an expression vector, and introduce the vector into a host cell.

In some embodiments, a host cell can express two or more modified enzymes. For example, a host cell can be transfected with a nucleic acid encoding two or more modified enzymes. Alternatively, a host cell can be transfected with more than one nucleic acid, each of which encodes one or more modified enzyme.

The nucleic acid encoding a modified enzyme can contain additional nucleic acid sequences. For example, the nucleic acid can contain a protein tag, a selectable marker, or a regulatory sequence that control the expression of the proteins in a host cell, such as promoters, enhancers or other expression control elements that control the transcription or translation of the nucleic acids (e.g., polyadenylation signals). Such regulatory sequences are known in the art. Those skilled in the art would appreciate that the choice of expression vector, including the selection of a regulatory sequence, may depend on several factors, including the choice of the host cell to be transformed, the level of expression of protein desired, etc. Exemplary regulator sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma virus.

In certain embodiments, a nucleic acid sequence containing a modified enzyme derived from GMD, FX, and/or FUT is introduced into a host cell. The fucosylation pathway will be changed, inhibited, or reduced in a host cell that expresses a modified enzyme.

c. Host Cells Expressing Modified Enzymes

Another aspect of the present disclosure relates to a host cell that expresses a modified enzyme in the fucosylation pathway. The expression of the modified enzyme in the host cell interferes with the activity of the wild-type enzyme, which results in the inhibition or reduction of the fucosylation pathway. Thus, proteins (e.g., antibodies) produced in a host cell that expresses the modified enzyme are afucosylated.

The phrase “low fucosylation cell” or “low fucosylation host cell”, as used herein, refers to a cell in which the fucosylation pathway has been inhibited or reduced because the cell expresses a modified enzyme in the fucosylation pathway.

A low fucosylation cell can be prepared by transfecting a host cell with an expression vector containing a nucleic acid sequence that encodes a modified enzyme in the fucosylation pathway. Transfection can be carried out using techniques known in the field. For example, transfection can be carried out using chemical-based methods (e.g., lipids, calcium phosphate, cationic polymers, DEAE-dextran, activated dendrimers, magnetic beads, etc.), by instrument-based methods (e.g., electroporation, biolistic technology, microinjection, laserfection/optoinjection, etc.), or by virus-based methods.

Transfected cells can be selected and isolated from non-transfected cells using a selectable marker present on the expression vector. In addition, transfected cells having an inhibited or reduced fucosylation pathway can be further selected and isolated from cells having a normal fucosylation pathway by various techniques. For example, fucosylation can be determined using antibodies, lectins, metabolic labeling, or chemoenzymatic strategies. In addition, cells having an inhibited or reduced fucosylation pathway can be selected by exposing the transfected cells to Lens culinaris agglutinin (LCA, Vector laboratories L-1040). LCA recognizes the α-1,6-fucosylated trimannose-core structure of N-linked oligosaccharides and commits cell expressing this structure to a cell-death pathway. Thus, cells that survive exposure to LCA have an inhibited or reduced fucosylation pathway, and are considered low fucosylation cells.

### 2. Afucosylated Proteins

Another aspect of the present disclosure relates to a method for producing afucosylated proteins. In some embodiments, the afucosylated protein is an afucosylated antibody.

a. Proteins

Non-limiting examples of proteins that can be produced as afucosylated proteins include GP-73, Hemopexin, HBsAg, hepatitis B viral particle, alpha-acid-glycoprotein, alpha-1-antichymotrypsin, alpha-1-antichymotrypsin His-Pro-less, alpha-1-antitrypsin, Serotransferrin, Ceruloplasmin, alpha-2-macroglobulin, alpha-2-HS-glycoprotein, alpha-fetoprotein, Haptoglobin, Fibrinogen gamma chain precursor, immunoglobulin (including IgG; IgA, IgM, IgD, IgE, and the like), APO-D, Kininogen, Histidine rich glycoprotein, Complement factor 1 precursor, complement factor I heavy chain, complement factor I light chain, Complement C1s, Complement factor B precursor, complement factor B Ba fragment, Complement factor B Bb fragment, Complement C3 precursor, Complement C3 beta chain, Complement C3 alpha chain, C3a anaphylatoxin, Complement, C3b alpha′ chain, Complement C3c fragment, Complement C3dg fragment, Complement C3g fragment, Complement C3d fragment, Complement C3f fragment, Complement C5, Complement C5 beta chain, Complement C5 alpha chain, C5a anaphylatoxin, Complement C5 alpha′ chain, Complement C7, alpha-1 B glycoprotein, B-2-glycoprotein, Vitamin D-binding protein, Inter-alpha-trypsin inhibitor heavy chain H2, Alpha-1B-glycoprotein, Angiotensinogen precursor, Angiotensin-1, Angiotensin-2-Angiotensin-3, GARP protein, beta-2-glycoprotein, Clusterin (Apo J), Integrin alpha-8 precursor glycoprotein, Integrin alpha-8 heavy chain, Integrin alpha-8 light chain, hepatitis C viral particle, elf-5, kininogen, HSP33-homolog, lysyl endopeptidase and Leucine-rich repeat-containing protein 32 precursor.

b. Antibodies

The term “antibody”, as used herein, broadly encompasses intact antibody molecules as well as fragments thereof that are capable of being fucosylated. For example, an antibody includes fully assembled immunoglobulins (e.g., polyclonal, monoclonal, monospecific, polyspecific, chimeric, deimmunized, humanized, human, primatized, single-chain, single-domain, synthetic, and recombinant antibodies); portions of intact antibodies that have a desired activity or function (e.g., immunological fragments of antibodies that contain Fab, Fab′, F(ab′)2, Fv, scFv, single domain fragments); as well as peptides and proteins that contain an Fc domain capable of being fucosylated (e.g., Fc-fusion proteins).

The term “afucosylated antibody”, as used herein, refers to an antibody or fragment thereof that is produced under conditions where fucosylation is inhibited or significantly reduced compared to antibodies produced under natural conditions. Afucosylated antibodies produced by methods of the present disclosure may be completely (100%) afucosylated or, alternatively, may comprise a mixture of fucosylated and afucosylated molecules. For example, in some embodiments, antibodies produced from the disclosed methods may contain from about 20% to about 100% afucosylated molecules. In other embodiments, the antibodies produced from the disclosed methods may contain from about 40% to about 100% afucosylated molecules. In certain embodiments, antibodies produced from the disclosed methods contain about at least 20%, 30%, 40%, 50%, 60%, 70%, 80%, 90%, 91%, 92%, 93%, 94%, 95%, 96%, 97, 98%, 99%, or 100% afucosylated molecules. It is not required that all the N-glycosylated antibodies or fragments thereof (e.g., Fc-fusion proteins) are afucosylated.

b. Types of Antibodies

Any antibody can be produced as an afucosylated antibody using the methods disclosed herein. There is no limitation on the types of antibodies that can be produced using the disclosed methods. The following is a non-exhaustive list of antibodies that can be produced:

Examples of antibodies that recognize a tumor-related antigen include anti-GD2 antibody, anti-GD3 antibody, anti-GM2 antibody, anti-HER2 antibody, anti-CD52 antibody, anti-MAGE antibody, anti-HM124 antibody, anti-parathyroid hormone-related protein (PTHrP) antibody, anti-basic fibroblast growth factor antibody and anti-FGF8 antibody, anti-basic fibroblast growth factor receptor antibody and anti-FGFS receptor antibody, anti-insulin-like growth factor antibody, anti-insulin-like growth factor receptor antibody, anti-PMSA antibody, anti-vascular endothelial cell growth factor antibody, anti-vascular endothelial cell growth factor receptor antibody and the like.

Examples of antibodies that recognize an allergy- or inflammation-related antigen include anti-interleukin 6 antibody, anti-interleukin 6 receptor antibody, anti-interleukin 5 antibody, anti-interleukin 5 receptor antibody and anti-interleukin 4 antibody, anti-tumor necrosis factor antibody, anti-tumor necrosis factor receptor antibody, anti-CCR4 antibody, anti-chemokine antibody, anti-chemokine receptor antibody and the like.

Examples of antibodies that recognize a circulatory organ disease-related antigen include anti-GpIIb/IIIa antibody, anti-platelet-derived growth factor antibody, anti-platelet-derived growth factor receptor antibody and anti-blood coagulation factor antibody and the like.

Examples of antibodies that recognize a viral or bacterial infection-related antigen include anti-gpl 20 antibody, anti-CD4 antibody, anti-CCR4 antibody and anti-Vero toxin antibody and the like.

Several therapeutic antibodies are commercially available, such as antibodies that bind to VEGF (e.g., Bevacizumab (AVASTIN®)), EGFR (e.g., Cetuximab (ERBITUX®)), HER2 (e.g., Trastuzumab (HERCEPTIN®)), and CD20 (e.g., Rituximab (RITUXAN®)), and Fc-fusion proteins that bind to TNFa (e.g., Etanecept (ENBREL®), which comprises the receptor-binding domain of a TNF receptor (p75)), CD2 (e.g., Alefacept (AMEVIVE®), which contains the CD2-binding domain of LFA-3), or B7 (Abatacept (ORENCIA®), which comprises the B7-binding domain of CTLA4).

### 3. Methods for Producing Afucosylated Proteins

Afucosylated proteins, including afucosylated antibodies, of the present disclosure are produced in a low fucosylation cell. Afucosylated proteins can be expressed in a low fucosylation cell using techniques known in the field, for example, by transfecting low fucosylation cells with an expression vector that encodes the protein.

An expression vector encoding a protein can prepared using techniques known in the field. For example, an expression vector can be constructed by reverse translating the amino acid sequence into a nucleic acid sequence, preferably using optimized codons for the organism in which the protein will be expressed. The nucleic acid encoding the protein, and any other regulatory elements, can then be assembled and inserted into the desired expression vector. The expression vector can contain additional nucleic acid sequences, such as a protein tag, a selectable marker, or a regulatory sequence that control the expression of the proteins, as described above for expression vectors containing the modified enzyme. The expression vector can then be introduced into a host cell by transfection. Transfection can be carried out using techniques known in the field. For example, transfection can be carried out using chemical-based methods (e.g., lipids, calcium phosphate, cationic polymers, DEAE-dextran, activated dendrimers, magnetic beads, etc.), by instrument-based methods (e.g., electroporation, biolistic technology, microinjection, laserfection/optoinjection, etc.), or by virus-based methods. The protein can then be expressed in the transfected cell under conditions appropriate for the selected expression system and host. The expressed protein can then be purified using an affinity column or other technique known in the field.

A host cell can be transfected with a nucleic acid encoding a modified enzyme (to become a low fucosylation cell) and a nucleic acid encoding a protein (to express the protein) in any order, to produce an afucosylated protein. For example, a host cell can be transfected with a nucleic acid encoding a modified enzyme (to become a low fucosylation cell) first and then transfected with a nucleic acid encoding an protein (to express the protein). Alternatively, a host cell can be transfected with a nucleic acid encoding a protein (to express the protein) first and then transfected with a nucleic acid encoding a modified enzyme (to become a low fucosylation cell). In another variation, a host cell can be transfected with a nucleic acid encoding a modified enzyme (to become a low fucosylation cell) and a nucleic acid encoding a protein (to express the protein) at the same time.

In a specific embodiment, an afucosylated protein is produced by first preparing a low fucosylation cell and then transfecting the low fucosylation cell with a nucleic acid encoding a protein according to the following steps:
a) obtaining host cells appropriate for expressing a protein;
b) transfecting the host cells with a nucleic acid encoding a modified enzyme;
c) selecting and/or isolating transfected cells having low fucosylation;
d) transfecting the low fucosylation cells with a nucleic acid encoding a protein;
e) selecting and/or isolating low fucosylation cells transfected with the nucleic acid encoding the protein;
f) inducing expression of the protein in the low fucosylation cells.

In a separate embodiment, an afucosylated protein is produced by transfecting a host cell with a nucleic acid encoding a protein first and then transfecting the cell with a nucleic acid encoding a modified enzyme according to the following steps:
a) obtaining host cells appropriate for expressing a protein;
b) transfecting the host cells with a nucleic acid encoding a protein;
c) selecting and/or isolating cells transfected with the nucleic acid encoding the protein;
d) transfecting the cells in step (c) with a nucleic acid encoding a modified enzyme;
e) selecting and/or isolating the transfected cells in step (d) having low fucosylation;
f) inducing expression of the protein in the low fucosylation cells.

In a variation of the above embodiment, an afucosylated protein is produced by:
a) obtaining host cells that express or over-express a protein;
b) transfecting the host cells with a nucleic acid encoding a modified enzyme;
c) selecting and/or isolating the transfected host cells having low fucosylation;
d) inducing expression of the protein in the low fucosylation cells.

In yet another embodiment, an afucosylated protein is produced by simultaneously transfecting a host cell with a nucleic acid encoding a modified enzyme (to become a low fucosylation cell) and a nucleic acid encoding a protein (to express the protein) as follows:
a) obtaining host cells appropriate for expressing a protein;
b) transfecting the host cells with a first nucleic acid encoding a protein and a second nucleic acid encoding a modified enzyme;
c) selecting and/or isolating the transfected host cells that express the protein and have low fucosylation;
d) inducing expression of the protein in the low fucosylation cells.

Afucosylated proteins, including antibodies, produced using the methods described above can be purified using methods known in the field. For example, afucosylated proteins, including antibodies, produced by the disclosed methods can be purified by physiochemical fractionation, antibody class-specific affinity, antigen-specific affinity, etc.

### 4. Improved Properties of Afucosylated Antibodies

The afucosylated antibodies produced by the method of the present disclosure have improved properties compared to antibodies produced using standard methods.

The activity of purified afucosylated antibodies can be measured by the ELISA and fluorescence method and the like. The cytotoxic activity for antigen-positive cultured cell lines can be evaluated by measuring its ADCC and CDC and the like. The safety and therapeutic effect of the antibody in human can be evaluated using an appropriate model of an animal species relatively close to human.

a. Increased ADCC Activity

Afucosylated antibodies of the present disclosure have increased ADCC activity compared to antibodies produced using standard methods.

“ADCC activity”, as used herein, refers to the ability of an antibody to elicit an antibody-dependent cellular cytotoxicity (ADCC) reaction. ADCC is a cell-mediated reaction in which antigen-nonspecific cytotoxic cells that express FcRs (e.g., natural killer (NK) cells, neutrophils, and macrophages) recognize antibodies bound to the surface of a target cell and subsequently cause lysis of (i.e., “kill”) the target cell. The primary mediator cells in ADCC are natural killer (NK) cells. NK cells express FcγRIII, with FcγRIIIA being an activating receptor and FcγRIIIB an inhibiting receptor. Monocytes express FcγRI, FcγRII and FcγRIII. ADCC activity can be assessed directly using an in vitro assay, such as the assay described in Example 3.

ADCC activity can be assessed directly using an in vitro assay. In some embodiments, the ADCC activity of afucosylated antibodies of the disclosure is at least 0.5, 1, 2, 3, 5, 10, 20, 50, 100 folds higher than that of the wild-type control itself.

Since afucosylated antibodies have an increased ADCC activity, therapeutic antibodies that are afucosylated can be administered in lower amounts or concentrations compared to their fucosylated counterparts. In some embodiments, the concentration of an afucosylated antibody of the present disclosure can be lowered by at least 2, 3, 5, 10, 20, 30, 50, or 100 fold compared to its fucosylated counterpart. In some embodiments, an afucosylated antibody of the present disclosure may exhibit a higher maximal target cell lysis compared to its wild-type counterpart. For example, the maximal target cell lysis of an afucosylated antibody of the present disclosure may be 10%, 15%, 20%, 25%, 30%, 40%, 50% or higher than that of its wild-type counterpart.

b. Increased CDC Activity

Afucosylated antibodies of the present disclosure have increased complement-dependent cytotoxicity (CDC) activity compared to antibodies produced using standard methods.

“CDC activity”, as used herein, refers to the reaction of one or more components of the complement system that recognizes bound antibody on a target cell and subsequently causes lysis of the target cell. Afucosylated antibodies of the present disclosure do not reduce or suppress CDC activity but, instead, they maintain CDC activity similar to, or greater than, its fucosylated counterpart.

The present invention further provides afucosylated antibodies with enhanced CDC function. In one embodiment, the Fc variants of the invention have increased CDC activity. In another embodiment said afucosylated antibodies have CDC activity that is at least 2 fold, or at least 3 fold, or at least 5 fold or at least 10 fold or at least 50 fold or at least 100 fold greater than that of a comparable molecule.

### 4. Using Afucosylated Antibodies

Afucosylated antibodies of the present disclosure can be administered intravenously (i.v.), subcutaneously (s.c.), intra-muscularly (i.m.), intradermal (i.d.), intraperitoneal (i.p.), or via any mucosal surface, e.g., orally (p.o.), sublingually (s.l. ), buccally, nasally, rectally, vaginally, or via pulmonary route.

Afucosylated antibodies are useful for treating or preventing various diseases including cancers, inflammatory diseases, immune and autoimmune diseases, allergies, circulator organ diseases (e.g., arteriosclerosis), and viral or bacterial infections.

The dose of the afucosylated antibodies of the invention will vary depending on the subject and the particular mode of administration. The required dosage will vary according to a number of factors known to those skilled in the art, including, but not limited to, the antibody target, the species of the subject and, the size/weight of the subject. Dosages may range from 0.1 to 100,000 μg/kg body weight. The afucosylated antibodies can be administered in a single dose or in multiple doses. The afucosylated antibodies can be administered once in a 24-hour period, multiple times during a 24-hour period, or by continuous infusion. The afucosylated antibodies can be administered continuously or at specific schedule. The effective doses can be extrapolated from dose-response curves obtained from animal models.

### 4. Specific Embodiments

Specific embodiments of the present invention include, but are not limited to, the following:
(1) A recombinant cell having low fucosylation that comprises a nucleic acid sequence encoding a modified enzyme of the fucosylation pathway.
(2) The recombinant cell according to (1), wherein the modified enzyme is derived from GDP-mannose 4,6-dehydratase (GMD), GDP-4-keto-6-deoxy-D-mannose epinierase-reductase (FX), or fucosyltransferase (FUT).
(3) The recombinant cell according to (1), wherein the modified enzyme is derived from fucosyltransferase (FUT).
(4) The recombinant cell according to (1), wherein the modified enzyme is derived from α-1,6-facosyltransferase (FUT8).
(5) The recombinant cell according to (1), wherein the nucleic acid sequence is selected from the group consisting of SEQ ID NOs: 3, 5, 7, 9, 11, 15, and any combination thereof.
(6) The recombinant cell according to (1), wherein the modified enzyme has an amino acid sequence selected from the group consisting of SEQ ID NOs: 4, 6, 8, 10, 12, 16, and any combination thereof.
(7) The recombinant cell according to (1), wherein the modified enzyme reduces or inhibits the activity of the wild-type enzyme, from which the modified enzyme is derived, in the host cell.
(8) The recombinant cell according to (1), wherein the modified enzyme inhibits or reduces fucosylation in the host cell.
(9) The recombinant cell according to (1), wherein less than 10% of the proteins produced in the cell are fucosylated.
(10) The recombinant cell according to (1) further comprising a nucleic acid encoding an antibody.

(11) The recombinant cell according to (10), wherein the antibody is expressed in the cell as an afucosylated antibody.
(12) An afucosylated antibody produced in the recombinant cell of (11).
(13) The afucosylated antibody according to (12) that is at least 90% afucosylated.
(14) The afucosylated antibody according to (12), wherein the afucosylated antibody has increased antibody-dependent cellular cytotoxicity (ADCC) activity compared to its fucosylated counterpart.
(15) The afucosylated antibody according to (12), wherein the complement dependent cytotoxicity (CDC) activity of the afucosylated antibody is not reduced or suppressed compared to its fucosylated counterpart.

### 5. Additional Embodiments

Additional embodiments of the present invention include, but are not limited to, the following:
(1) A method for producing an afucosylated antibody comprising:

introducing a nucleic acid encoding at least one modified enzyme to a host cell to produce the afucosylated antibody in the host cell.
(2) The method according to (1), wherein the modified enzyme is derived from GDP-mannose 4,6-dehydratase (GMD), GDP-4-keto-6-deoxy-D-mannose epinierase-reductase (FX), or fucosyltransferase (FUT).
(3) The method according to (1), wherein the modified enzyme is derived from fucosyltransferase (FUT).
(4) The method according to (1), wherein the modified enzyme is derived from α-1,6-fucosyltransferase (FUT8).
(5) The method according to (1), wherein the modified enzyme inhibits the activity of the wild-type fucosylation enzymes in the host cell.
(6) The method according to (1), wherein the modified enzyme inhibits and/or reduces the fucosylation of antibody in the host cell.
(7) The method according to (1), wherein the afucosylated antibody has increased ADCC.
(8) The method according to (1), wherein the CDC activity of the afucosylated antibody is not reduced or suppressed.
(9) A method for producing an afucosylated antibody comprising:

a) providing a host cell,

b) introducing a nucleic acid encoding at least one modified enzyme to the host cell and

c) producing an afucosylated antibody in the host cell.
(10) The method according to (9), wherein in step (a), the host cell comprises at least one nucleic acid encoding an antibody.
(11) The method according to (9), further introducing a nucleic acid encoding an antibody to the host cell after the step (b).
(12) The method according to (9), wherein the modified enzyme is derived from GDP-mannose 4,6-dehydratase (GMD), GDP-4-keto-6-deoxy-D-mannose epinierase-reductase (FX), and/or fucosyltransferase (FUT).
(13) The method according to (9), wherein the modified enzyme is derived from fucosyltransferase (FUT).
(14) The method according to (9), wherein the modified enzyme is derived from α-1,6-fucosyltransferase (FUT8).
(15) The method according to (9), wherein the modified enzyme inhibits the activity of the fucosylation enzymes in the host cell.
(16) The method according to (9), wherein the modified enzyme inhibits and reduces the glycosylation of antibody in the host cell.
(17) The method according to (9), wherein the afucosylated antibody has increased ADCC.
(18) The method according to (9), wherein the CDC activity of the afucosylated antibody is not reduced or suppressed.
(19) An afucosylated antibody produced by a method according to (1) or (9), wherein the afucosylated antibody has increased ADCC activity.
(20) The antibody according to (19), wherein the afucosylated antibody is a human antibody or a fragment thereof.
(21) The antibody according to (19), wherein the afucosylated antibody maintain the original CDC activity.
(22) A pharmaceutical composition comprising the afucosylated antibody according to (19) and a pharmaceutically acceptable carrier or excipient.
(23) A cell without or with low-fucosylation comprising a nucleic acid encoding at least one modified enzyme.
(24) An afucosylated antibody produced by:

a) introducing a nucleic acid encoding at least one modified enzyme of the fucosylation pathway to a host cell expressing an antibody with fucose, and

b) culturing the host cell to produce the afucosylated antibody in the host cell.
(25) The afucosylated antibody according to (24), wherein the modified enzyme is derived from GDP-mannose 4,6-dehydratase (GMD), GDP-4-keto-6-deoxy-D-mannose epinierase-reductase (FX), or Fucosyltransferase (FUT).
(26) The afucosylated antibody according to (25), wherein the modified enzyme is derived from fucosyltransferase.
(27) The afucosylated anti-CD20 antibody according to (26), wherein the modified enzyme is derived from α-1,6-fucosyltransferase.
(28) The afucosylated anti-CD20 antibody according to (24), wherein the modified enzyme inhibits the activity of the wild-type fucosylation enzymes in the host cell.
(29) The afucosylated anti-CD20 antibody according to (24), wherein the modified enzyme inhibits and reduces the glycosylation of antibody in the host cell.
(30) The afucosylated anti-CD20 antibody according to (24), wherein the afucosylated antibody has increased antibody-dependent cellular cytotoxicity (ADCC) activity.
(31) The afucosylated anti-CD20antibody according to (24), wherein the complement dependent cytotoxicity (CDC) activity of the afucosylated antibody is not reduced and suppressed.
(32) The afucosylated anti-CD20 antibody according to (24), wherein the afucosylated antibody is an anti-CD20 or anti-ErbB2 antibody.
(33) An afucosylate antibody produced by:

a) providing a host cell,

b) introducing a nucleic acid encoding at least one modified enzyme of the fucosylation pathway to the host cell,

c) introducing a nucleic acid encoding an antibody with fucose, and

d) producing an afucosylated antibody in the host cell.
(34) The afucosylated antibody according to (33), wherein the modified enzyme is derived from GDP-mannose 4,6-dehydratase (GMD), GDP-4-keto-6-deoxy-D-mannose epinierase-reductase (FX), and/or Fucosyltransferase (FUT).
(35) The afucosylated antibody according to (33), wherein the modified enzyme is derived from α-1,6-fucosyltransferase.
(36) The afucosylated antibody according to (33), wherein the antibody has increased antibody-dependent cellular cytotoxicity (ADCC).
(37) The afucosylated antibody according to (33), wherein the complement dependent cytotoxicity (CDC) activity of the afucosylated antibody is not reduced and suppressed.
(38) The afucosylated antibody according to (33), wherein the afucosylated antibody is an anti-CD20 or anti-ErbB2 antibody.
(39) A pharmaceutical composition comprising the afucosylate antibody according to claim 1 or 10 and a pharmaceutically acceptable carrier or excipient

Additional specific embodiments of the present invention include, but are not limited to the following examples.

### EXAMPLE 1

### Preparation of a Modified Enzyme in the Fucosylation Pathway and Stable Cell Lines Expressing the Modified Enzyme

### 1. Cell Lines

The commercial CHOdhfr⁽⁻⁾ cell line (ATCC CRL-9096), which is a CHO cell mutant deficient in dihydrofolate reductase activity, was purchased from Culture Collection and Research Center (CCRC, Taiwan). The CHOdhfr⁽⁻⁾ cell line was separated into three separate cultures and treated as follows:

The first culture was transfected with an expression vector encoding RITUXAN® (Rituximab, a chimeric monoclonal antibody against the protein CD20). A stable clone expressing RITUXAN® was obtained and identified as RC79.

The second culture was transfected with and expression vector encoding HERCEPTIN® (Trastuzumab, a monoclonal antibody against the protein HER2). A stable clone expressing HERCEPTIN® was obtained and identified as HC59.

The third culture was left untreated and maintained as a CHOdhfr(−) cell line.

### 2. Construction of Expression Vectors Encoding Modified Enzymes of FUT8 and GMD

Several expression vectors encoding modified enzymes FUT8 and GMD were constructed.

The mutants of F83M, F8M1, F8M2, F8M3, and F8D1 represent different modifications of α-1,6-fucosyltransferase, the wild-type FUT8 protein (GenBank No. NP_058589.2). Table 1 summarizes the modifications that were made to the wild-type nucleic acid sequence for each FUT8 vector as well as resulting amino acid changes in the expressed enzyme. Specifically, F83M represents a mutant that has three modifications in the wild-type FUT8 protein at R365A, D409A, and D453A. F8M1, F8M2, and F8M3 represent mutants that have one modification each at K369E, D409K, and S469V in wild-type FUT8 protein, respectively. F8D1 represents a mutant that has a deletion of an amino acid residues at position 365 to 386 in wild-type FUT8 protein.

Table 2 summarizes the modifications that were made to the wild-type nucleic acid sequence for the GMD vector as well as resulting amino acid changes in the expressed enzyme. Specifically, the mutant GMD4M represents a modification of GDP-mannose 4,6-dehydratase, the wild-type GMD protein (GenBank No. NP_001233625.1), that has four mutations in the wild-type GMD protein at T155A, E157A, Y179A, and K183A.

All of nucleic acid sequences encoding F83M, F8M1, F8M2, F8M3, F8D1, and GMD4M were synthesized by GeneDireX company, and then subcloned into the PacI/EcoRv or BamHI/EcoRV site of pHD expression vector (pcDNA3.1Hygro, Invitrogen, Carlsbad, Calif., cat. no. V870-20 with dhfr gene) to form pHD/F83M, pHD/F8M1, pHD/F8M2, pHD/F8M3, pHD/F8D1, and pHD/GMD4M plasmids.

3. Preparation of Stable Recombinant Cell Lines that Express a Modified Enzyme

The pHD/F83M, pHD/F8M1, pHD/F8M2, pHD/F8M3, pHD/F 8D 1 , and pHD/GMD4M plasmids were transfected into different cell lines, including (a) a RC79 cell line (CHO cell expressing RITUXAN®), (b) a HC59 cell line (CHO cell expressing HERCEPTIN®), and (c) CHOdhfr⁽⁻⁾ cells (CHO cell mutants deficient in dihydrofolate reductase activity) by electroporation (PA4000 PULSEAGILE® electroporator, Cyto Pulse Sciences).

a. RC79 Cells

The transfected RC79 cell lines were initially cultured in RC79 culture medium (EX-CELL® 302 serum free medium containing 0.4 μM MTX, 0.5 mg/mL Geneticin, 0.05 mg/mL Zeocin, 4 mM Glutamax-I, and 0.01% F-68) with 0.1 to 0.25 mg/mL Hygromycin. Then, the transfected cells were cultured in EX-CELL® 302 serum free medium containing 0.4 μM MTX, 0.5 mg/mL Geneticin, 0.05 mg/mL Zeocine, 4 mM Glutamax-I, 0.01% F-68, and 0.25 mg/mL Hygromycin and isolated by Lens culinaris agglutinin (LCA), as described below, to generate five cell pools including RC79F83M, RC79F8M1, RC79F8M2, RC79F8M3, RC79F8D1, and RC79-GMD4M cell lines.

b. HC59 Cells

The transfected HC59 cell lines were initially cultured in HC59 culture medium (EX-CELL® 325 PF CHO Medium containing 0.8 μM MTX, 0.5 mg/mL Geneticin, 0.05 mg/mL Zeocine, and 4 mM Glutamax-I) with 0.1 to 0.25 mg/mL Hygromycin. Then, the transfected cells were cultured in EX-CELL® 325 PF CHO medium containing 0.8 μM MTX, 0.5 mg/mL Geneticin, 0.05 mg/mL Zeocine, 4 mM Glutamax-I, and 0.25 mg/mL Hygromycin and isolated by LCA, as described below, to generate a cell pools of HC59F83M cell line.

c. CHOdhfr⁽⁻⁾ Cells

The transfected CHOdhfr⁽⁻⁾ cell lines were initially cultured in EX-CELL® 325 PF CHO Medium containing 4 mM Glutamax-I, and 0.1 to 0.25 mg/mL Hygromycin. Then, the transfected cells were cultured in EX-CELL® 325 PF CHO medium containing 4 mM Glutamax-I, 0.25 mg/mL Hygromycin, and 0.01 μM MTX to generate a cell pools of C109F83M cell line.

4. Isolation of Cells with Low-Fucosylation

Rhodamine-labeled Lens Culinaris Agglutinin (LCA) (Vector Laboratories, Cat. RL-1042) was used in this Example to select the cells with low fucosylation.

All RC79, HC59, and CHO transfectants were subjected to primary selection medium containing Hygromycin as a selection pressure followed by final selection using LCA, which recognizes the α-1,6-fucosylated trimannose-core structure of N-linked oligosaccharides and commits cell expressing this structure to a cell-death pathway. Transfectants of RC79, HC59, or CHO were seeded at 1.2×10⁵ cells/mL in 2.5 mL fresh medium with 0.4 mg/mL LCA initially and counted on day 3 or 4 for cell viability. The cells were cultured in this initial selection medium until the cell viability reached 80%. After the cell viability reached 80%, the cells were resuspended in fresh selection medium with gradually increasing concentrations of LCA at 1.2×10⁵ cells/mL. The LCA selection was repeated several times, until a final concentration of LCA of 0.6-1.2 mg/mt, was achieved.

To analyze the fucose level on the cell surface, the cells were labeled with LCA and analyzed by flow-cytometry. First, cells were seeded in complete medium without LCA for 14 days to remove signal interference from the selection agent LCA. Then, 3×10⁵ cells were washed with 1 mL ice cold PBS twice, and resuspended in 200 μl cold PBS containing 1% bovine serum albumin and 5 μg/mL LCA. After incubation on ice for 30 min, the cells were washed with 1 mL ice cold PBS twice. The cells were resuspended in 350 μl cold PBS and analyzed using a FACScalibur™ flow cytometer (BI) Biosciences, San Jose, Calif.).

Next, 1×10⁷ cells were washed with 10 mL ice cold PBS twice, and resuspended in 6.5 mL ice cold PBS containing 1% bovine serum albumin and 5 μg/mL LCA. After incubation on ice for 30 min, the cells were washed with 10 mL cold PBS twice. The cells were resuspended in 1 mL ice cold PBS with 1% heat-inactivated fetal bovine serum (GIBCO, Cat. 10091-148) and Antibiotic-Antimycotic (Invitrogen, Cat. 15240062).

The cells were analyzed and sorted by FACSAria™ or Influx™ Cell Sorter (BD Biosciences, San Jose, Calif.). For different clones, 1-3 rounds of sorting were necessary to generate a homogenous population of cells with low fucosylation levels. In addition, stable clones with low-fucosylation were isolated using a CLONEPIX™ 2 system (MOLECULAR DEVICES®) and transferred to 96-well plates. After culturing for approximately two weeks, the cells were transferred to 6-well plates and analyzed again by flow-cytometry. Cells with low-fucosylation were then transferred to a filter tube for fed-batch culture to evaluate cell performance and fucosylation level of the antibody purified from the obtained cells.

### 5. Preparation of C109F83M Cell Line to Express RITUXAN®

After the low fucosylation CHOdhfr⁽⁻⁾ cells (C109F83M cells) were isolated by LCA, the cells were transfected with a nucleic acid encoding RITUXAN® by electroporation (PA4000 PULSEAGILE® electroporator, Cyto Pulse Sciences). Low-fucose single clone of C109F83M, AF97, was isolated and transfected with a nucleic acid encoding RITUXAN® by electroporation for expressing RITUXAN®. The transfectant was transfer to 25 T flask containing non-selective medium for recovery growth. After 48 hr the transfectants were cultured under selective medium containing 4 mM GlutaMAX-I, Hygromycin-B, Zeocin and 0.01 μM MTX. A single cell was picked using the CLONEPIX™ 2 System to generate the AF97anti-CD20 clone.

The obtained cells were low fucosylation CHOdhfr(−) cells that express RITUXAN® and are referred to herein as the AF97anti-CD20 cell line.

### EXAMPLE 2

### Expression and Analysis of Afucosylated Antibodies

### 1. Expression and Purification of Antibody

Cells with low-fucosylation activity obtained in Example 1 were cultured in batch or fed-batch for antibody expression. Antibodies purified from the cells were subjected to a monosaccharide analysis for quantitation analysis of the sugar chains in the Fc regions.

Recombinant RC79 cells were cultured in EX-CELL® 302 serum free medium containing 4 mM Glutamax and 0.01% F-68, and maintained in shaker incubator (Infors Multitron Pro) with 37° C. and 5% CO₂.

Recombinant HC79 cells were cultured in EX-CELL® 325 PF CHO medium containing 0.8 μM MTX, 0.5 mg/mL Geneticin, 0.05 mg/mL Zeocine, 4 mM Glutamax-I, and 0.25 mg/mL Hygromycin, and maintained in shaker incubator (Infors Multitron Pro) with 37° C. and 5% CO₂.

The parameters of cell culture were routinely monitored every day. Cell density and viability were determined by trypan blue exclusion using a hemocytometer. When cell viability was below 60%, the conditioned medium was collected by centrifugation and the expressed antibodies were purified with protein A resin. Protein A column was equilibrated with 0.1 M Tris, pH 8.3 for 5 column volume and then load sample into column. The unbound proteins were washed out with 0.1 M Tris, 8.3 (for 2 column volume) and PBS, pH 6.5 (for 10 column volume). The column was further washed with 0.1 M sodium acetate, pH 6.5 (for 10 column volume). Finally, the antibodies were eluted with 0.1 M glycine, pH 2.8 and neutralized with 0.1 M Tris, pH 8.3 for equal elution volume.

### 2. Determination of N-Glycan Profile of Antibody

The N-glycan profile was analyzed by ACQUITY UPLC® System. First, 0.3 mg antibody sample was digested with 3 U PNGase-F in 0.3 mL digestion buffer (15 mM Tris-HCl, pH 7.0) at 37° C. for 18 hr. The released N-glycans were separated from the antibody by ultrafiltration using an AMICON® Ultra-0.5 mL 30K device at 13,000 rpm for 5 min and then freeze-dried for 3 hr. Next, the dried N-glycans were dissolved in 30 μL ddH₂O and 45 μL 2-AB labeling reagent (0.34 M Anthranilamide and 1 M sodium cyanoborohydride in DMSO-acetic acid (7:3 v/v) solvent) and incubated at 65° C. for 3 hr. Excess 2-AB labeling reagent was removed with a PD MINITRAP™ G10 size exclusion column. The labeled N-glycans were freeze-dried overnight and re-dissolved in 50 μL ddH₂O for UPLC detection. The N-glycan profiles were acquired by ACQUITY UPLC® System with Glycan BEH Amide Column at 60° C. The different forms of N-glycans were separated with 100 mM ammonium formate, pH 4.5/acetonitrile linear gradient.

The results of flow cytometry revealed extremely low binding of LCA on the surface of cells over-expressing the F83M protein in all cell types. Similarly, LCA binding was not detected on the RC79 cells over-expressing F8M1, F8M2, F8M3, F8D1, or GMD4M protein (data not shown).

Table 3 shows the N-glycan profile of antibodies produced in RC79 and HC59 cells having an unmodified fucosylation pathway as well as RC79 and HC59 clones whose fucosylation pathway were modified by over-expressing the F83M modified enzyme. The data in Table 3 show that most of the anti-CD20 and anti-ErbB2 antibodies produced in the cells having an unmodified fucosylation pathway were heavily fucosylated. Specifically, only 3.67% of the anti-CD20 and 3.64% of the anti-ErbB2 antibodies were afucosulated in these cells. In contrast, antibodies produced in the cells over-expressing the F83M modified enzyme had very low fucosylation levels. Specifically, about 98.86-98.91% of the anti-CD20 and about 92.12-96.52% of the anti-ErbB2 antibodies were afucosylated in the cells over-expressing the F83M modified enzyme.

In addition, Table 4 shows the N-glycan profile of antibodies produced in RC79 cells having an unmodified fucosylation pathway as well as RC79 clones whose fucosylation pathway were modified by over-expressing one of the F8M1, F8M2, F8M3, F8D1, or GMD4M modified enzymes. The data in Table 4 show that most of the anti-CD20 antibodies produced in the RC79 cells having an unmodified fucosylation pathway were heavily fucosylated. Specifically, only 3.67% of the anti-CD20 antibodies were afucosulated in these cells. In contrast, antibodies produced in the RC79 cells over-expressing a modified enzyme had very low fucosylation levels. Specifically, the afucosylation level of anti-CD20 antibodies produced by cells over-expressing F8M1, F8M2, F8M3, F8D1, or GMD4M modified enzyme was between about 92.78% to about 97.16%, as shown in Table 4.

Table 4 also shows that the afucosylation level of antibody produced by cells over-expressing one of the FUT8 modified enzymes (F8M1, F8M2, F8M3, F8D1) was between 95.70 to 97.16%, and the afucosylation level of antibody produced by cells over-expressing GMD modified enzyme (GMD4M) was 92.78%. These results demonstrate that the afucosylation level of an antibody produced in cells over-expressing a FUT8 modified enzyme was higher than that of an antibody produced in cells over-expressing GMD mutant protein.

The results shown in Tables 3 and 4 demonstrate that host cells that have been engineered to express antibodies can be transfected with a vector expressing a modified enzyme in the fucosylation pathway (FUT8 or GMD). The results also show that antibodies produced in these transfected cells are afucosylated.

In addition, the fucosylation level of antibodies produced in the AF97 cell line was evaluated. The results in Table 5 show that the antibodies produced in the AF97 cells over-expressing the F83M modified enzyme had very low fucosylation levels. Specifically, 97.83% of the anti-CD20 antibody produced in the AF97 cells were afucosylated. In contrast, commercial RITUXAN® (MABTHERA®) had an afucosylation level of 3.92%.

The results in Table 5 demonstrate that afucosylated antibodies can be produced in cells that are transfected first with a nucleic acid encoding a modified enzyme and then transfected a second time with a nucleic acid encoding an antibody. Therefore, cells can be modified using the disclosed methods to produce afucosylated antibodies.

### 3. FUT8 Protein Expression in Recombinant Cell

The pellet of RC79 cells and recombinant cells that express the FUT8 modified enzyme (i.e., F8M1, F8M2, F8M3, or F8D1) were lysed in 1% Triton X-100 containing a phosphatase inhibitor cocktail (Sigma-Aldrich, Cat. S8820). The protein concentration in the supernatants of the lysed cells were determined by DC™ (detergent compatible) protein assay (BIO-RAD). The supernatants, containing 30 μg of protein for each sample, were separated using 12.5% sodium dodecyl sulfate-polyacrylamide gel electrophoresis (SDS-PAGE) and transferred onto nitrocellulose membranes. The membranes were blocked for 1 h at room temperature by using 25 mM Tris-HCl (pH7.4) containing 120 mM NaCl, 0.1% gelatin (w/w) and 0.1% TWEENER® 20 (polyethylene glycol sorbitan monolaurate) (v/w) and incubated overnight at 4° C. with anti-FUT8 antibody (Abcam, Cat. ab204124, 1:500) and GAPDH antibody (GeneTex, Cat. GT239, 1:10000), respectively. The membranes were washed 3 times for 5 min with 25 mM Tris-HCl (pH7.4) containing 120 mM NaCl, 0.1% gelatin (w/w) and 0.1% TWEEN® 20 (v/w), and then incubated with goat anti-rabbit IgG (Jackson ImmunoResearch, Cat. 111-035-144) and goat anti-mouse IgG HRP (GeneTex, Cat. GTX213111-01, 1:10000), respectively, for 1 h at room temperature. Following additional washes, the membranes were analyzed with SIGMAFAST DAB with Metal Enhancer (Sigma, Cat. D0426).

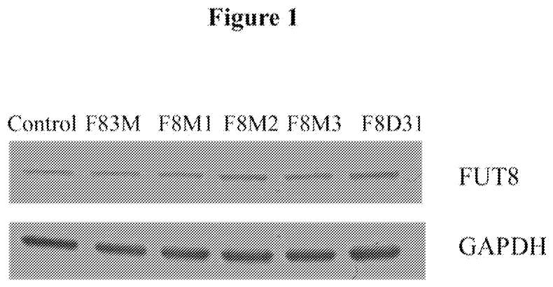

FIG. 1 is a western blot that shows the FUT8 protein expression in the RC79 parent cells and the RC79 recombinant cells expressing a modified enzyme. The expression level of FUT8 protein was similar in the recombinant cells and the parent RC79 cells. The results indicate that the production of afucosylated antibody produced in the RC79 cells expressing a modified enzyme was not related to the expression level of the FUT8 protein. These results suggest that the FUT8 modified enzymes interfere with the wild-type FUT8 protein in cells to inhibit and/or reduce the fucosylation pathway so that the recombinant cells produce afucosylated antibody efficiently.

The mechanism of producing afucosylated antibodies using the disclosed methods is novel and unique compared to other methods that rely on suppressing or down-regulating the wild-type FUT8 gene or utilize RNA interference to reduce the expression of FUT8 protein.

### 4. Stability of Recombinant Cell

The stability of the RC79 recombinant cells expressing the F83M modified enzyme was evaluated.

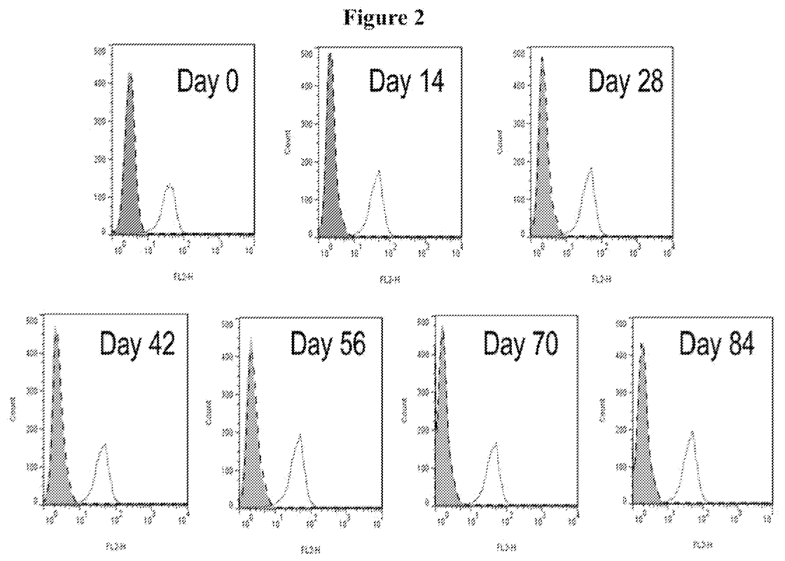

The RC79 recombinant cells were cultured in medium without selection reagent for three months. Cellular fucosylation was monitored by flowcytometry analysis every week and the composition of N-glycan of purified antibody was determined by ACQUITY UPLC® System with Glycan BEH Amide Column every month for three months, as described above. The LCA non-binding properties were maintained over the 90-day evaluation period, indicating that the fucosylation pathway was inhibited and/or reduced over the course of the study (FIG. 2).

In addition, the afucosylation level of anti-CD20 antibodies produced in five stable RC79F83M clones (R4-R8) was evaluated over a 72-day period. As shown in Table 6, all of the RC79F83M clones produced highly afucosylated antibodies over the 72-day study.

The results from this study demonstrate that recombinant cell lines expressing a modified enzyme prepared by the disclosed methods are stable and produce highly-afucosylated antibody for a long period of time.

### EXAMPLE 3

### ADCC Activity of Afucosylated Antibodies

In order to evaluate in vitro cytotoxic activity of the purified anti-CD20 obtained from Example 2, the ADCC activity was measured in accordance with the following method.

### 1. Preparation of Effect Cell Solution

Human peripheral blood from healthy donors (100 mL) was added to VACUTAINER® tubes containing sodium heparin. The whole blood sample was diluted at 1:1 with RPMI 1640 serum free (SF) medium and mix gently. The mononuclear cells were separated using Ficoll-Paque PLUS by smoothly applying 24 mL of the diluted blood onto the Ficoll-Paque and centrifuging at 400×g for 32 min at 25° C. The buffy coat was adequately distributed into two of 50 mL centrifuge tube containing 20 mL of RPMI 1640 medium and then mixed two times. Then the mixture was centrifuged at 1,200 rpm for 12 min at 25° C. to obtain the supernatant. RPMI 1640 SF medium (13 mL) was added to the supernatant to re-suspend the PBMC cells. The cells were centrifuged at 1,200 rpm for 12 min at 25° C. to obtain the supernatant. RPMI culture medium (10 mL) was added to the supernatant to re-suspend the PBMC cells. An adequate volume of PBMC cell suspension was added to a 75 T flask and the final cell density was 1.5×10⁶ cells/mL for about 15 mL per flasks. IL-2 (2.5 μg/mL) was added to all flasks at a final concentration of 3 ng/mL. The PBMC cells were incubated in a 37° C., 5% CO₂ incubator for 18 hrs. IL-2 stimulated PBMC cells were collected and centrifuged at 1,200 rpm for 5 min at 25° C. and then the supernatant was discarded. PBS (10 mL) was added and mixed with the cells. The cells were centrifuged at 1,200 rpm for 5 min at 25° C. to remove supernatant. The cells were re-suspended with RPMI and the final concentration was adjusted to 2×10⁷ cells/mL.

### 2. Preparation of Target Cell Solution

The cell suspension from 75 T flasks was centrifuged at 1,000 rpm for 5 min to remove the supernatant and then washed with 10 mL of 1× PBS. The washed cells were centrifuged at 1,200 rpm for 5 min to remove the supernatant. The cells were re-suspended by RPMI assay medium to prepare 5×10⁵ cells/mL target cell solution. The target cell solution (40 μL of 5×10⁵ cells/mL) was added to the wells of the V-bottomed 96-well cell culture plate. Then, 20 μL of prepared commercial RITUXAN® solution (MABTHERA®) (25-0.0025 μg/mL) (positive control), afucosylated antibody (R1 clone) solution (25-0.0025 μg/mL), or RPMI assay medium (negative control) were added to the wells and mixed with target cell solution, respectively. The V-bottomed 96-well cell culture plates were incubated in a 37° C., 5% CO₂ incubator for 30 to 60 min.

### 3. ADCC Activity Assay

The effector cell solution (40 μL of 8×10⁵ effector cells/well) or 40 μL of RPMI assay medium was added to the plates to mix with target cell solution. The plates were centrifuged at 300×g for 4 min. The plates were incubated at 37° C., 5% CO₂ for 4 hr. Lysis solution (10 μL) of CYTOTOX 96® was added to the plates of Tmax and BlkV groups for one hour before harvesting the supernatant. V-bottomed 96-well cell culture plate was centrifuged at 300×g for 4 min, and the 50 μL of the supernatant was transferred to the wells of flat-bottomed assay plate from 96-well cell culture plates.

Lactate dehydrogenase (LDH) (2 μL) was added to 10 mL of LDH positive control diluent to prepare LDH positive control solution. Prepared LDH positive control solution (50 μL) was added to wells of 96-well flat-bottomed assay plate.

LDH reconstitute substrate mix (50 μL) was added to each test well of the assay plates. The plates were covered and incubated at room temperature in dark for 30 min. Stop solution (50 μL) was added to each test well of the plates. The absorbance at 490 nm was recorded immediately after the addition of the stop solution. Blank-removed absorbance values of each group (S, PBMC, T, E, and Tmax) was used to calculate ADCC activity by the formula listed below.

$$ADCC activity ( \% ) = \frac{S ( or PBMC ) - E - T}{Tmax - T} \times 100 \%$$

where S is the absorbance value of LDH release of the sample (target cell+PBMC+anti-CD20 antibody); PBMC is the absorbance value of LDH release of the target cell and PBMC; E is the absorbance value of LDH release of PBMC; T is the absorbance value of the target cell spontaneous LDH release; and Tmax is the absorbance value of the target cell maximum LDH release.

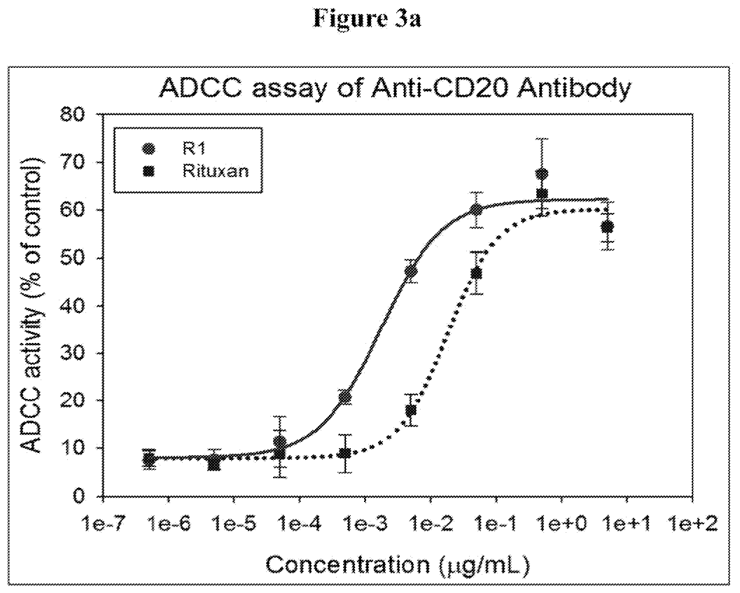

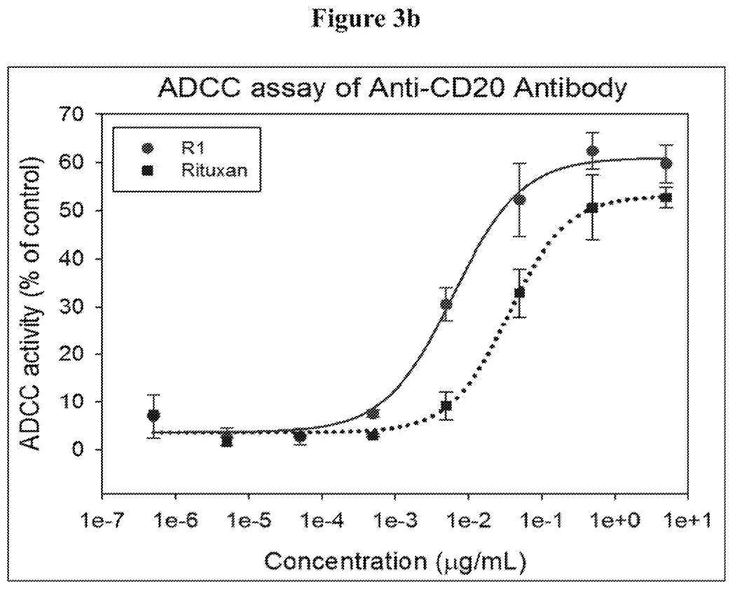

The afucosylated anti-CD20 antibody (clone R1) induced a significantly stronger and higher ADCC response in PBMC cells from both donor 1 (FIG. 3a) and donor 2 (FIG. 3b) compared to commercial RITUXAN® (MABTHERA®).

As shown in Table 7, the EC₅₀ of the afucosylated anti-CD20 antibody from the RC79F83M clone R1 was significantly lower than the EC₅₀ of the commercial RITUXAN®, which is a fucosylated anti-CD20 antibody. Specifically, the afucosylated anti-CD20 antibody (clone R1) had an EC₅₀ of 1.7 ng/mL and 4.6 ng/mL in PBMC cells from donors 1 and 2, respectively. In contrast, the fucosylated anti-CD20 antibody (MABTHERA®)) had an EC₅₀ of 18.2 ng/mL and 35.0 ng/mL in PBMC cells from donors 1 and 2, respectively.

The results from this study demonstrate that afucosylated anti-CD20 antibody (clone R1) exhibited between 7.68-fold to 10.7-fold stronger ADCC activity than fucosylated anti-CD20 antibody (MABTHERA®).

### EXAMPLE 4

### Binding Affinity of Afucosylated Antibody

The binding affinity of afucosylated and fucosylated anti-CD20 antibodies to His-tagged FcγRIIIa recombinant protein was evaluated using anti-histidine (anti-His) antibody coupled to a BIACORE® CM5 chip with amine coupling kit and the immobilization wizard of BIACORE® X100 control software.

His-tagged FeγRIIIa recombinant protein (1 μg/mL) was injected onto anti-His antibody-immobilized CM5 chip at the flow rate of 10 μL/min for 20 seconds.

Afucosylated anti-CD20 antibody from clone 1 (5, 10, 20, 40, and 80 nM), a commercial fucosylated anti-CD20 antibody RITUXAN® (MABTHERA®) (20, 40, 80, 160, and 320 nM), and a commercial afucosylated anti-CD20 antibody GAZYVA® (obinutuzumab) (5, 10, 20, 40, or 80 nM) were injected through the chips at the flow rate of 30 μL/min for 3 min, respectively. The running buffer flowed through the chips at the flow rate of 30 μL/min for 5 min. Glycine, pH 1.5 (10 mM) was injected to the chips at the flow rate of 30 μL/min for 60 seconds.

The sensorgram of each cycle was analyzed with RIACORE® X100 evaluation software to obtain the value of equilibrium dissociation constant (K_{D}), association rate constant (Ka), and dissociation rate constant (Kd). The sensorgram of each cycle was fitted by 1:1 Langmuir binding model. If Chi² value was lower than 1/10× Rmax value, the fitting model was adequate and the kinetic binding parameters were reliable.

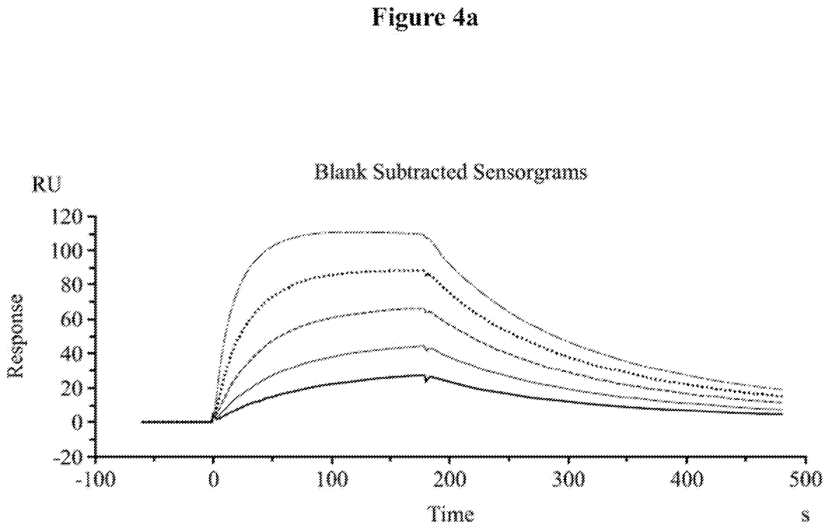

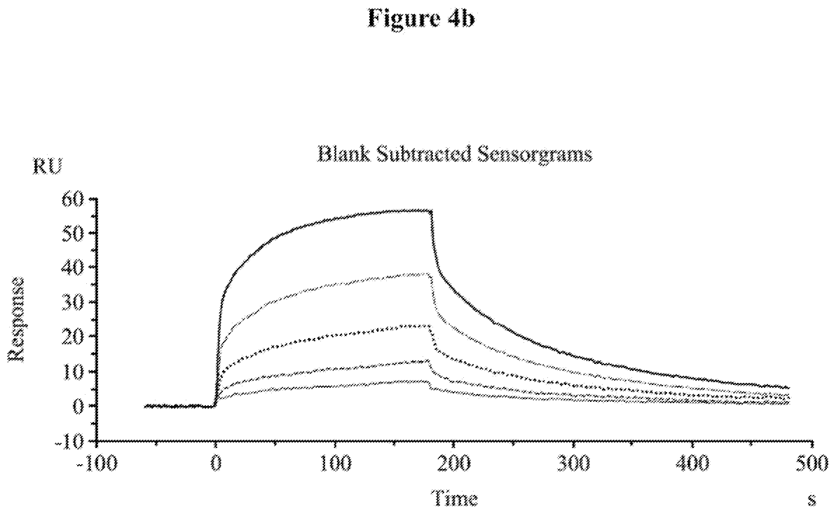

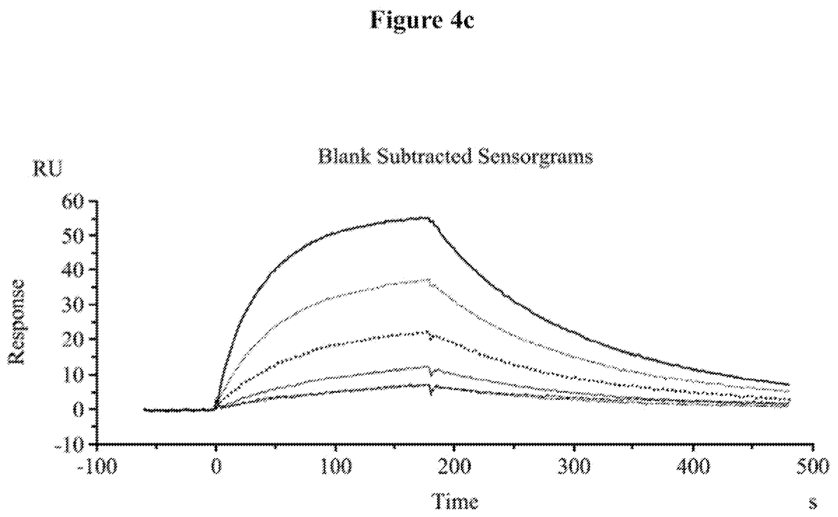

FIGS. 4a-4c show the classic SPR sensorgrams of the three antibodies tested. The classic SPR sensorgrams indicated that the conditions used in this assay (e.g., association time, dissociation time, and antibody concentration range) were adequate. In addition, the Chi² values of the three antibodies were smaller than 1/10× Rmax values, which indicated that 1:1 Langmuir model was suitable for the sensorgram fitting of all three antibodies.

As shown in Table 8, the afucosylated anti-CD20 antibody (clone R1) had more than a 10-fold stronger binding affinity to FcγRIIIa compared to MABTHERA® (K_{D} of R1 clone=13.0 nM, MABTHERA®=151.5 nM). Additionally, the afucosylated anti-CD20 antibody (clone R1) had more than a 3-fold stronger binding affinity to FcγRIIIa compared to GAZYVA® (K_{D} of R1 clone=13.0 nM, GAZYVA®=39.9 nM).

The results from this study demonstrate that the afucosylated anti-CD20 antibody (clone R1), prepared according to the present disclosure, has a greater FcγRIIIa binding affinity compared to a commercial fucosylated anti-CD20 antibody RITUXAN® (MABTHERA®) as well as the commercial afucosylated anti-CD20 antibody (GAZYVA®).

### EXAMPLE 5

### CDC Activity of Afucosylated Antibody

The CDC activity of afucosylated antibodies produced by the disclosed methods was evaluated.

Daudi cells were cultured with RPMI culture medium and sub-cultured when the cell density reached 1×10⁶ cells/mL (subculture density: 2-3×10⁵ cells/mL). The Daudi cells were collected and centrifuged at 300 rpm for 5 min. The cells were re-suspended with RPMI culture medium to prepare a cell suspension at a concentration of 1×10⁵ cells/mL. After resuspension, 100 μL of cell suspension or 100 μL of RPMI culture medium was seeded into the wells of white 96-well plates.

Commercially available RITUXAN® (MABTHERA®) and afucosylated anti-CD20 antibody (clone R1) were prepared in saline at concentrations between 120 μg/mL to 0.234 μg/mL. Then, 25 μL of RITUXAN® or afucosylated anti-CD20 antibody (clone R1) solution at 120 μg/mL to 0.234 μg/mL were added to the wells of white 96-well plates containing the Daudi cells or RPMI medium. CELLTITER-GLO® reagent (20 μL) was added to each well and then mixed. The plates were placed on a microplate shaker at 750 rpm for 2 min and then incubated at room temperature for 10 min in the dark. Luminescent intensity was detected by a multi-mode reader plugged with a high sensitivity luminescent cassette (integrate time: 1 second) to calculate EC₅₀ values of the anti-CD20 antibodies and the related CDC activity of the antibodies.

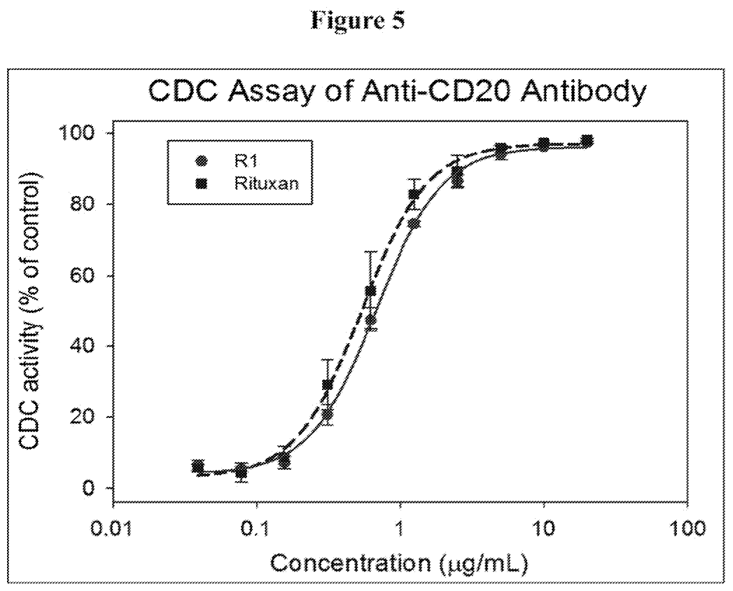

FIG. 5 shows that the CDC activity of afucosylated anti-CD20 antibody (clone R1) was comparable to that of RITUXAN®. The EC₅₀ value of afucosylated anti-CD20 antibody (R1 clone) was 0.682 μg/mL, which was higher than that of RITUXAN® (EC₅₀=0.582 μg/mL).

GAZYVA®, a commercial afucosylated anti-CD20 antibody, has been shown to induce ADCC activity, hut supress CDC activity (E. Mössener et al, (2010); C. Ferrara et al. (2011)). The results obtained by others suggest that the amount of GAZYVA® should be increased in order to obtain an efficient cancer treatment. In contrast, the results from this Example and Example 5 demonstrate that the afucosylated anti-CD20 antibody produced by the disclosed methods induces ADCC activity while maintaining CDC activity similar to its fucosylated counterpart. Therefore, the afucosylated anti-CD20 antibody of the present disclosure performed better than GAZYVA®.

### EXAMPLE 6

### Proof of Efficacy for the Afucosylated Antibody in Animal Models

B-cell lymphoma subcutaneous xenograft model was used in this Example to prove the antitumor efficacy of afucosylated antibody of the present disclosure, SU-DHL-4 is a B-cell lymphoma cell line expressing high level of CD20 on cell membrane and can grow and form a solid tumor subcutaneously. Thus, the xenograft model in SCID/Beige mice was developed to compare the antitumor efficacy of afucosylated antibody (R1 clone) and commercially available RITUXAN® (MABTHERA®).

SU-DHL-4 cells were cultured with the RPMI culture medium (CM) in flasks. When the cell concentration reached 0.8-1.0×10⁶ cells/mL, the cell suspension was collected and centrifuged at 300 g for 5 min to remove the supernatant. The cells were re-suspended with new culture medium containing some of the condition medium (the ratio of fresh CM:condition CM=9:1). The cells were sub-cultured at a ratio of 1:2 to 1:10 (seeding cell number:total harvest cell number), and the cell concentration was at least 1×10⁵ cells/mL. The culture dishes were incubated 37° C. SU-DHL-4 cells were cultured in five 150 T flasks. When the cell concentration reached 0.8 to 1.0×10⁶ cells/mL, the cell suspension was collected in 50 mL tubes and then centrifuged at 1,200 rpm for 5 min to remove the supernatant. The cell concentration was adjusted to 1×10⁸ cells/mL using serum free RPMI medium. The cell suspension was mixed with an equal volume of MATRIGEL® in a 50 mL-centritube using a pre-chilled syringe with 18 G needle on ice. The final cell concentration was 5×10⁷ cells/mL. Matrigel-SU-DHL-4 cells mixture (100 uL) at a concentration of 5×10⁷ cells/mL was subcutaneously injected at the right side of dorsal area of each mouse (SCID/Beige mouse) using a pre-chilled 1 mL syringe with a 23G*1″ needle. The total inoculation cell number was 5×10⁶ cells. The tumor volume of each mouse was measured using a caliper every 3 or 4 days, and calculated by the equation: V=0.5×ab² , where a and b are tumor length and width, respectively.

When the tumor volume reached about 200 mm³ (198.25±55.53 mm³), which occurred approximately 20 days after tumor inoculation, the mice were distributed into three groups of five, and then the treated with saline (vehicle), commercially available RITUXAN® (MABTHERA®), or afucosylated anti-CD20 antibody (clone R1). The mice were injected with 0.2 mL of 0.1 mg/mL antibody weekly for 3 weeks. The body weight and tumor size of all mice were measured twice weekly by an electronic scale and a digital caliper. At the end of treatment period, the mice were sacrificed and the tumor tissues were removed and weighed. The tumor tissues were then fixed in 10% formalin buffer at room temperature for further examination.

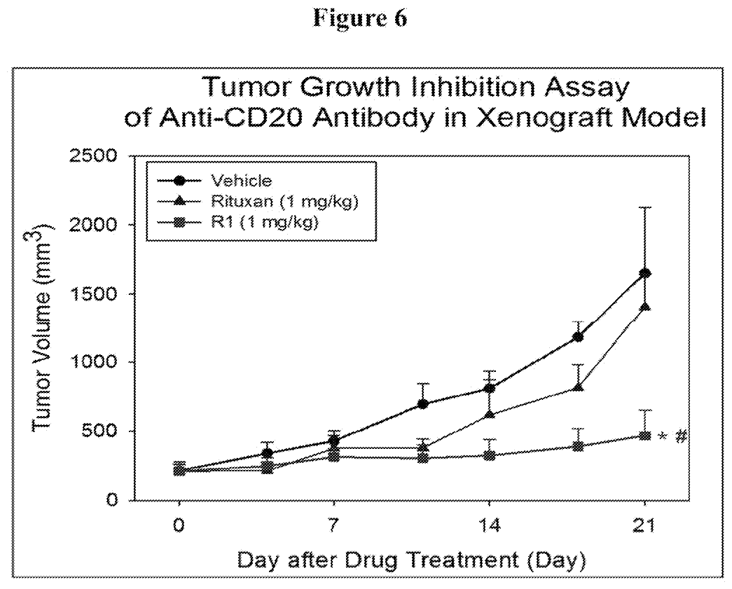

As shown in FIG. 6, the afucosylated anti-CD20 antibody (clone R1) showed significantly stronger anti-tumor efficacy than RITUXAN®. There was statistically significant difference (P<0.001, by student t-test) in tumor volume between the vehicle group and the group treated with afucosylated anti-CD20 antibody (clone R1). On the contrary, RITUXAN® did not show a statistically significant difference in tumor volume when compared to the vehicle-only group. The afucosylated anti-CD20 antibody (clone R1) suppressed tumor growth more effectively compared to RITUXAN® at a dose of 1 mg/kg (R1 clone=468±148 mm³; RITUXAN®=1407±241 mm³) as shown in Table 9. There was statistically significant difference in tumor volume between afucosylated anti-CD20 antibody (clone R1) and RITUXAN® groups (P<0.001).

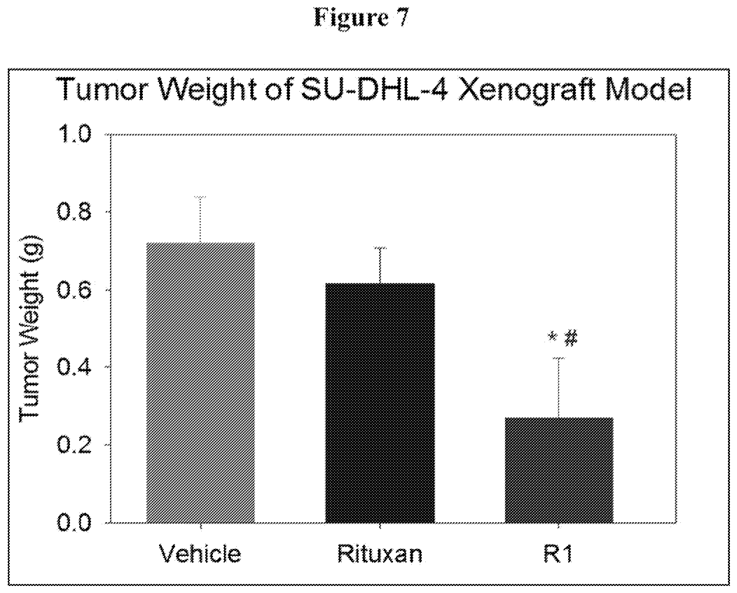

The tumor weight of the group treated with afucosylated anti-CD20 antibody (R1 clone) was significantly less than that of the vehicle-only group (P<0.001), as shown in FIG. 7. However, RITUXAN® did not show a statistically significant difference in tumor weight at the same dose in comparison to vehicle group. Thus, there was a statistically significant decrease in tumor weight in the afucosylated anti-CD20 antibody (clone R1) group compared to RITUXAN® group (R1 clone=0.27±0.15 g; RITUXAN®=0.62±0.09 g, P<0.01), as shown in Table 9. The afucosylated anti-CD20 antibody (clone R1) inhibited tumor growth much more efficiently than RITUXAN®, which corresponds to the results obtained with the tumor volume.

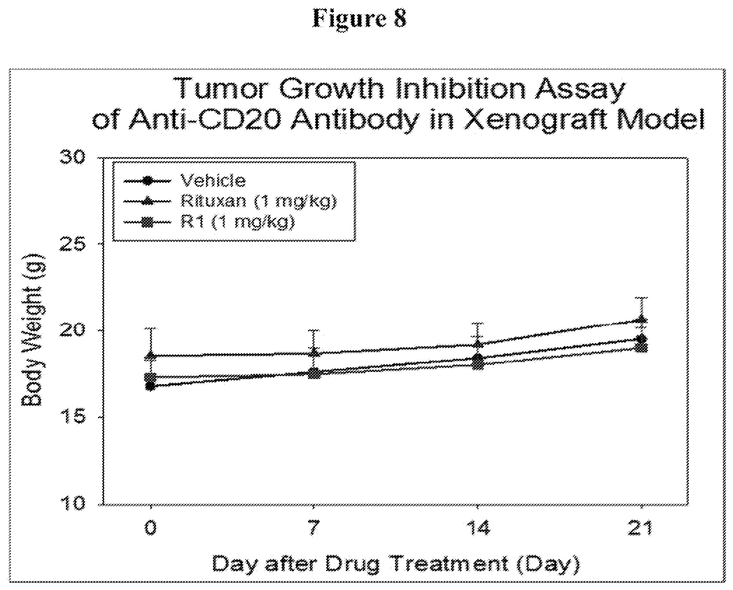

The body weight of the mice in all groups gradually increased during treatment period, as shown in FIG. 8.

The results from the studies suggest that afucosylated anti-CD20 antibody (clone R1) was safe.

TABLE 1
Modifications of the FUT8 Enzyme Employed in Assays

| | DNA | | | DNA | Amino Acid | Amino Acid |
|---|---|---|---|---|---|---|
| Code | location¹ | Wild type | Mutation | SEQ ID NO | location² | SEQ ID NO |
| F83M | 1093-1095 | AGG | GCC | 3 | R365A | 4 |
| | 1225-1227 | GAC | | | D409A | |
| | 1357-1359 | GAC | | | D453A | |
| F8M1 | 1105-1107 | AAG | GAG | 5 | K369E | 6 |
| F8M2 | 1225-1227 | GAC | AAG | 7 | D409K | 8 |
| F8M3 | 1405-1407 | TCC | GTG | 9 | S469V | 10 |
| F8D1 | 1087-1149 | CACGTGAGGAGGACCGACAA | N/A³ | 11 | Deletion of amino | 12 |
| | | GGTGGGCACCGAGGCCGCCT | | | acid residues from | |
| | | TCCACCCCATCGAGGAGTAC | | | 365 to 386 | |
| | | ATG | | | | |
| | | (SEQ ID NO: 17) | | | | |

¹DNA sequence location based on the wild-type FUTS DNA sequence of SEQ ID NO: 1
²Amino acid location based on the wild-type FUT8 protein sequence of SEQ ID NO: 2
³N/A = Not Applicable

TABLE 2
Modifications of the GMD Enzyme Employed in Assays

| | | | | DNA | Amino | Amino/ |
|---|---|---|---|---|---|---|
| | DNA | Wild | Muta- | SEQ | Acid | Acid SEQ |
| Code | location¹ | type | tion | ID NO | location² | ID NO |
| GMD4M | 463-465 | ACC | GCC | 15 | T155A | 16 |
| | 469-471 | GAG | | | E157A | |
| | 535-537 | TAC | | | Y179A | |
| | 547-549 | AGG | | | K183A | |

¹DNA sequence location based on the wild-type sequence of SEQ ID NO: 13
²Amino acid location based on the wild-type sequence of SEQ ID NO: 14

TABLE 3
N-glycan Profile of Antibodies Produced in CHO Cells With and Without Over-Expression of the F83M Modified Enzyme

| | | | | | | | | | | | | w/o Fuc. |
|---|---|---|---|---|---|---|---|---|---|---|---|---|
| | | G0-GN | G0F-GN | G0 | G0F | Man5 | G1 | G1F | Man6 | G2 | G2F | (%) |
| RITUXAN ® | MABTHERA ® | 0.11 | 0.72 | 0.71 | 42.36 | 1.26 | 1.16 | 44.35 | 0.11 | 0.32 | 8.89 | 3.67 |
| (anti-CD20) | RC79F83M | 1.36 | 0.00 | 58.17 | 0.66 | 1.60 | 33.35 | 0.33 | 0.24 | 4.13 | 0.15 | 98.86 |
| | Clone R1 | | | | | | | | | | | |
| | RC79F83M | 1.37 | 0.00 | 57.22 | 0.68 | 1.49 | 34.18 | 0.16 | 0.29 | 4.31 | 0.31 | 98.85 |
| | Clone R2 | | | | | | | | | | | |
| | RC79F83M | 1.28 | 0.00 | 59.20 | 0.62 | 2.48 | 31.62 | 0.30 | 0.26 | 4.06 | 0.17 | 98.91 |
| | Clone R3 | | | | | | | | | | | |
| HERCEPTIN ® | HERCEPTIN ® | 0.13 | 0.73 | 0.82 | 42.07 | 1.32 | 1.15 | 44.04 | 0.22 | n/a | 9.50 | 3.64 |
| (anti-ErbB2) | HC59F83M | 5.29 | 0 | 65.23 | 3.65 | 17.68 | 3.72 | 1.85 | 2.18 | n/a | 0.39 | 94.11 |
| | Clone H1 | | | | | | | | | | | |
| | HC59F83M | 4.13 | 0 | 68.92 | 2.00 | 17.41 | 4.16 | 1.13 | 1.91 | n/a | 0.35 | 96.52 |
| | Clone H2 | | | | | | | | | | | |
| | HC59F83M | 3.07 | 0 | 68.74 | 5.26 | 15.08 | 3.51 | 2.28 | 1.72 | n/a | 0.34 | 92.12 |
| | Clone H3 | | | | | | | | | | | |

w/o Fuc.: (amount of total glycan − amount of fucose)/amount of total glycan × 100%

TABLE 4
N-glycan Profile of Anti-CD20 Antibody Produced by Cells With and Without
Over-Expression of the F8M1, F8M2, F8M3, F8D1, or GMD4M Modified Enzyme

| | | | | | | | | | | | w/o Fuc. |
|---|---|---|---|---|---|---|---|---|---|---|---|
| | G0-GN | G0F-GN | G0 | G0F | Man5 | G1 | G1F | Man6 | G2 | G2F | (%) |
| MABTHERA ® | 0.11 | 0.72 | 0.71 | 42.36 | 1.26 | 1.16 | 44.35 | 0.11 | 0.32 | 8.89 | 3.67 |
| RC79F8M1 | 1.12 | 0.19 | 51.16 | 1.07 | 0.58 | 36.64 | 1.43 | 1.83 | 5.83 | 0.15 | 97.16 |
| RC79F8M2 | 1.80 | 0.34 | 56.53 | 1.78 | 0.60 | 31.57 | 1.92 | 1.29 | 3.91 | 0.26 | 95.70 |
| RC79F8M3 | 2.50 | 0.21 | 54.03 | 1.53 | 0.15 | 35.89 | 0.85 | 0.62 | 4.41 | 0.26 | 97.16 |
| RC79F8D1 | 1.27 | n/a | 57.47 | 2.30 | 2.03 | 29.99 | 1.54 | 0.24 | 4.04 | 0.43 | 95.70 |
| RC79GMD4M | 1.80 | 0.67 | 41.58 | 2.33 | 0.6 | 35.35 | 3.23 | 2.53 | 5.66 | 0.58 | 92.78 |

w/o Fuc.: (amount of total glycan − amount of fucose)/amount of total glycan × 100%

TABLE 5
N-glycan Profile of Anti-CR20 Antibody Produced by Cells With
and Without Over-Expression of the F83M Modified Enzyme

| | | | | | | | | | | | w/o Fuc. |
|---|---|---|---|---|---|---|---|---|---|---|---|
| | G0-GN | G0F-GN | G0 | G0F | Man5 | G1 | G1F | Man6 | G2 | G2F | (%) |
| MABTHERA ® | n/a | 0.78 | 0.74 | 41.93 | 1.41 | 1.09 | 43.90 | 0.29 | 0.39 | 9.48 | 3.93 |
| AF97antiCD20 | 4.50 | n/a | 62.00 | 0.51 | 10.62 | 16.32 | 1.25 | 2.88 | 1.51 | 0.41 | 97.83 |

w/o Fuc: (amount of total glycan − amount of fucose)/amount of total glycan × 100%

TABLE 6
Stability of Afucosylated Anti-CD20 Antibodies Produced
by Cells Over-Expressing F83M Modified Enzyme

| | Day 0 | Day 28 | Day 56 | Day 72 |
|---|---|---|---|---|
| MABTHERA ® | 3.67% | 5.34% | 3.42% | 3.67% |
| RC79F83M | 98.86% | 98.58% | 99.68% | 99.15% |
| Clone R4 | | | | |
| RC79F83M | 98.86% | 98.76% | 98.57% | 99.30% |
| Clone R5 | | | | |
| RC79F83M | 98.90% | 98.89% | 99.57% | 98.88% |
| Clone R6 | | | | |
| RC79F83M | 99.46% | 98.68% | 99.47% | 99.13% |
| Clone R7 | | | | |
| RC79F83M | 98.88% | 98.40% | 99.08% | 98.38% |
| Clone R8 | | | | |

TABLE 7
EC₅₀ Value and ADCC Activity of Fucosylated
and Afucosylated Anti-CD20 Antibodies

| | | |
|---|---|---|
| | EC₅₀ | Related ADCC activity |
| R square | (ng/mL) | (Fold) |

Donor 1

| | | | |
|---|---|---|---|
| MABTHERA ® | 0.9742 | 18.2 | 1.00 |
| RC79F83M | 0.9667 | 1.7 | 10.70 |
| Clone R1 | | | |

Donor 2

| | | | |
|---|---|---|---|
| MABTHERA ® | 0.9779 | 35.0 | 1.00 |
| RC79F83M | 0.9777 | 4.6 | 7.68 |
| Clone R1 | | | |

TABLE 8
FcγRIIIa Binding Affinity of RITUXAN ®, GAZYVA ®, and
Afucosylated Antibodies

| | Kₐ | K_{d} | K_{D} | Rmax | |
|---|---|---|---|---|---|
| | (M⁻¹S⁻¹) | (S⁻¹) | (nM) | (RU) | Chi² |
| MABTHERA ® | 4.9 × 10⁴ | 7.5 × 10⁻³ | 151.5 | 52.2 | 1.52 |
| GAZYVA ® | 17.3 × 10⁴ | 6.9 × 10⁻³ | 39.9 | 77.9 | 0.40 |
| Clone R1 | 48.5 × 10⁴ | 6.3 × 10⁻³ | 13.0 | 114.4 | 3.39 |

TABLE 9
Tumor Volume, Tumor Weight, and Body Weight of Treated Mice in Each Group

| | | | Tumor Volume | Tumor Weight | Body weight |
|---|---|---|---|---|---|
| Group | Name | Dose | (mm³) | (g) | (g) |
| 1 | Vehicle | — | 1652 ± 474 | 0.72 ± 0.12 | 19.5 ± 1.2 |
| 2 | MABTHERA ® | 1 mg/kg | 1407 ± 241 | 0.62 ± 0.09 | 20.7 ± 1.3 |
| 3 | Clone R1 | 1 mg/kg | 468 ± 184 | 0.27 ± 0.15 | 19.0 ± 1.2 |

Values represent means ± S.D. (S.C.)

1. A recombinant cell having low fucosylation that comprises a nucleic acid sequence encoding a modified enzyme of the fucosylation pathway.

2. The recombinant cell according to claim 1, wherein the modified enzyme is derived from GDP-mannose 4,6-dehydratase (GMD), GDP-4-keto-6-deoxy-D-mannose epinierase-reductase (FX), or fucosyltransferase (FUT).

3. The recombinant cell according to claim 1, wherein the modified enzyme is derived from fucosyltransferase (FUT).

4. The recombinant cell according to claim 1, wherein the modified enzyme is derived from α-1,6-fucosyltransferase (FUT8).

5. The recombinant cell according to claim 1, wherein the nucleic acid sequence is selected from the group consisting of SEQ ID NOs: 3, 5, 7, 9, 11, 15, and any combination thereof.

6. The recombinant cell according to claim 1, wherein the modified enzyme has an amino acid sequence selected from the group consisting of SEQ ID NOs: 4, 6, 8, 10, 12, 16, and any combination thereof.

7. The recombinant cell according to claim 1, wherein the modified enzyme reduces or inhibits the activity of the wild-type enzyme, from which the modified enzyme is derived, in the host cell.

8. The recombinant cell according to claim 1, wherein the modified enzyme inhibits or reduces fucosylation in the host cell.

9. The recombinant cell according to claim 1, wherein less than 10% of the proteins produced in the cell are fucosylated.

10. The recombinant cell according to claim 1 further comprising a nucleic acid encoding an antibody.

11. The recombinant cell according to claim 10, wherein the antibody is expressed in the cell as an afucosylated antibody.

12. An afucosylated antibody produced in the recombinant cell of claim 11.

13. The afucosylated antibody according to claim 12 that is at least 90% afucosylated.

14. The afucosylated antibody according to claim 12, wherein the afucosylated antibody has increased antibody-dependent cellular cytotoxicity (ADCC) activity compared to its fucosylated counterpart.

15. The afucosylated antibody according to claim 12, wherein the complement dependent cytotoxicity (CDC) activity of the afucosylated antibody is not reduced or suppressed compared to its fucosylated counterpart.
